(19) United States
(12) Patent Application Publication
(10) Pub. No.: US 2010/0184813 A1
(43) Pub. Date: Jul. 22, 2010

(54) CHEMICAL COMPOUNDS 553

(75) Inventors: Alan Martin BIRCH, Macclesfield (GB); Gary Peter Tomkinson, Macclesfield (GB); Sharmistha Pal, Bangalore (IN); Anna Pettersen, Molndal (SE)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 12/642,195

(22) Filed: Dec. 18, 2009

Related U.S. Application Data
(60) Provisional application No. 61/139,032, filed on Dec. 19, 2008.

(51) Int. Cl. A61K 31/4245; C07D 271/113; A61P 3/04; A61P 3/10

(52) U.S. Cl. 514/364; 548/143

(57) ABSTRACT

DGAT-1 inhibitor compounds of formula (I) and pharmaceutically-acceptable salts thereof are described, together with pharmaceutical compositions, processes for making them and their use in treating, for example, obesity

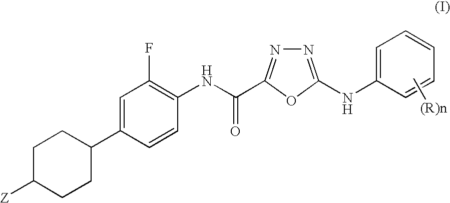

wherein n is 0, 1, 2 or 3, R is independently selected from fluoro, chloro, bromo, trifluoromethyl, methoxy, difluoromethoxy and trifluoromethoxy and
Z is carboxy or a mimic or bioisostere thereof, hydroxyl, hydroxymethyl, or —CONRbRc wherein Rb and Rc are independently selected from hydrogen and (1-4C)alkyl, which (1-4C)alkyl group may be optionally substituted by carboxy or a mimic or bioisostere thereof.

This application claims the benefit under 35 U.S.C. §119(e) of Application No. 61/139,032 (US) filed on 19 Dec. 2008.

The present invention relates to compounds which inhibit acetyl CoA(acetyl coenzyme A): diacylglycerol acyltransferase (DGAT1) activity, processes for their preparation, pharmaceutical compositions containing them as the active ingredient, methods for the treatment of disease states associated with DGAT1 activity, to their use as medicaments and to their use in the manufacture of medicaments for use in the inhibition of DGAT1 in warm-blooded animals such as humans. In particular this invention relates to compounds useful for the treatment of type II diabetes, insulin resistance, impaired glucose tolerance and obesity in warm-blooded animals such as humans, more particularly to the use of these compounds in the manufacture of medicaments for use in the treatment of type II diabetes, insulin resistance, impaired glucose tolerance and obesity in warm-blooded animals such as humans.

Acyl CoA: diacylglycerol acyltransferase (DGAT) is found in the microsomal fraction of cells. It catalyzes the final reaction in the glycerol phosphate pathway, considered to be the main pathway of triglyceride synthesis in cells by facilitating the joining of a diacylglycerol with a fatty acyl CoA, resulting in the formation of triglyceride. Although it is unclear whether DGAT is rate-limiting for triglyceride synthesis, it catalyzes the only step in the pathway that is committed to producing this type of molecule [Lehner & Kuksis (1996) Biosynthesis of triacylglycerols. Prog. Lipid Res. 35: 169-201].

Two DGAT genes have been cloned and characterised. Both of the encoded proteins catalyse the same reaction although they share no sequence homology. The DGAT1 gene was identified from sequence database searches because of its similarity to acyl CoA: cholesterol acyltransferase (ACAT) genes. [Cases et al (1998) Identification of a gene encoding an acyl CoA: diacylglycerol acyltransferase, a key enzyme in triacylglycerol synthesis. Proc. Natl. Acad. Sci. USA 95: 13018-13023]. DGAT1 activity has been found in many mammalian tissues, including adipocytes.

Because of the previous lack of molecular probes, little is known about the regulation of DGAT1. DGAT1 is known to be significantly up-regulated during adipocyte differentiation.

Studies in gene knockout mice has indicated that modulators of the activity of DGAT1 would be of value in the treatment of type II diabetes and obesity. DGAT1 knockout (Dgat1^{-/-}) mice, are viable and capable of synthesizing triglycerides, as evidenced by normal fasting serum triglyceride levels and normal adipose tissue composition. Dgat1^{-/-} mice have less adipose tissue than wild-type mice at baseline and are resistant to diet-induced obesity. Metabolic rate is ˜20% higher in Dgat1^{-/-} mice than in wild-type mice on both regular and high-fat diets [Smith et al (2000) Obesity resistance and multiple mechanisms of triglyceride synthesis in mice lacking DGAT. Nature Genetics 25: 87-90]. Increased physical activity in Dgat1^{-/-} mice partially accounts for their increased energy expenditure. The Dgat1^{-/-} mice also exhibit increased insulin sensitivity and a 20% increase in glucose disposal rate. Leptin levels are 50% decreased in the Dgat1^{-/-} mice in line with the 50% decrease in fat mass.

When Dgat1^{-/-} mice are crossed with ob/ob mice, these mice exhibit the ob/ob phenotype [Chen et al (2002) Increased insulin and leptin sensitivity in mice lacking acyl CoA: diacylglycerol acyltransferase J. Clin. Invest. 109:1049-1055] indicating that the Dgat1^{-/-} phenotype requires an intact leptin pathway. When Dgat1^{-/-} mice are crossed with Agouti mice a decrease in body weight is seen with normal glucose levels and 70% reduced insulin levels compared to wild type, agouti or ob/ob/Dgat1^{-/-} mice.

Transplantation of adipose tissue from Dgat1^{-/-} mice to wild type mice confers resistance to diet-induced obesity and improved glucose metabolism in these mice [Chen et al (2003) Obesity resistance and enhanced glucose metabolism in mice transplanted with white adipose tissue lacking acyl CoA: diacylglycerol acyltransferase J. Clin. Invest. 111: 1715-1722].

International Application WO 2006/064189 discloses certain oxadiazole compounds which inhibit DGAT1. However, there remains a need for further DGAT1 inhibitors possessing desirable properties, such as, for example, pharmaco-kinetic/dynamic and/or physico-chemical and/or toxicological profiles and/or selective activity for DGAT1.

The present invention provides a compound of formula (I), or a pharmaceutically-acceptable salt thereof,

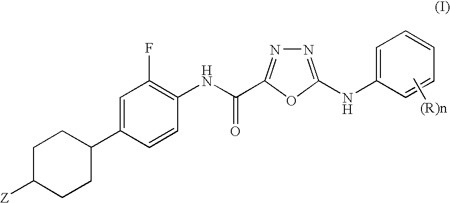

wherein n is 0, 1, 2 or 3, R is independently selected from fluoro, chloro, bromo, trifluoromethyl, methoxy, difluoromethoxy and trifluoromethoxy and
Z is carboxy or a mimic or bioisostere thereof, hydroxyl, hydroxymethyl, or —CONRbRc wherein Rb and Rc are independently selected from hydrogen and (1-4C)alkyl, which (1-4C)alkyl group may be optionally substituted by carboxy or a mimic or bioisostere thereof.

Also provided are carboxylic acid mimics or bioisosteres of the compounds of formula (I), or a pharmaceutically-acceptable salt thereof.

### BRIEF DESCRIPTION OF DRAWINGS

FIG. 1 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Form A

FIG. 2 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Form B

FIG. 3 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Form C

FIG. 4 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Form D

FIG. 5 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Form E

FIG. 6 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Form F

FIG. 7 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Nanosuspension Form B

FIG. 8 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Nanosuspension Form D

FIG. 9 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Nanosuspension Form F

FIG. 10 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Sodium salt

FIG. 11 shows the X-ray powder diffraction pattern for (1r,4r)-4-(4-(5-(3,4-Difluorophenylamino)-1,3,4-oxadiazole-2-carboxamido)-3-fluorophenyl)cyclohexanecarboxylic acid: Magnesium salt

As used herein, the reference to carboxylic acid mimic or bioisostere includes groups as defined in The Practice of Medicinal Chemistry, Wermuth C. G. Ed.: Academic Press: New York, 1996, p203. Particular examples of such groups include —SO₃H, —S(O)₂NHR¹³, S(O)₂NHC(O)R¹³, —CH₂S(O)₂R¹³, —C(O)NHS(O)₂R¹³, —C(O)NHOH, —C(O)NHCN, —CH(CF₃)OH, C(CF₃)₂OH, —P(O)(OH)₂ and groups of sub-formula (a)-(i′) below

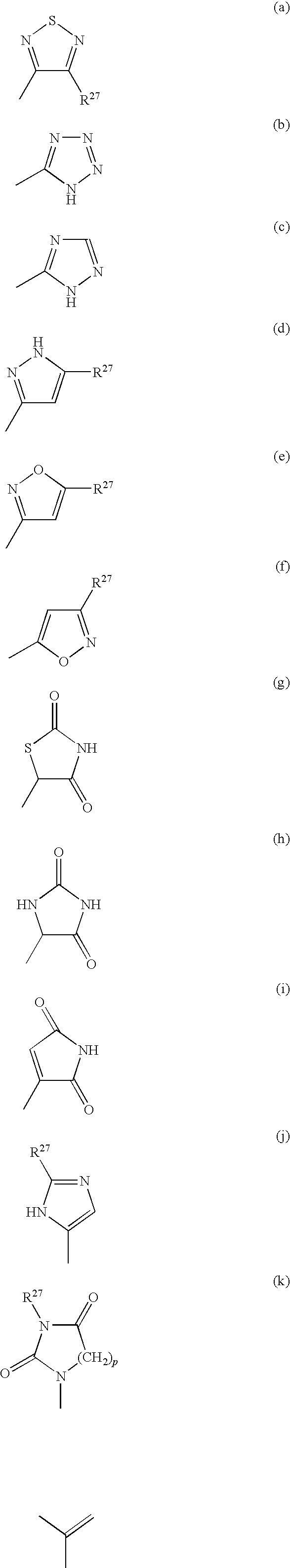

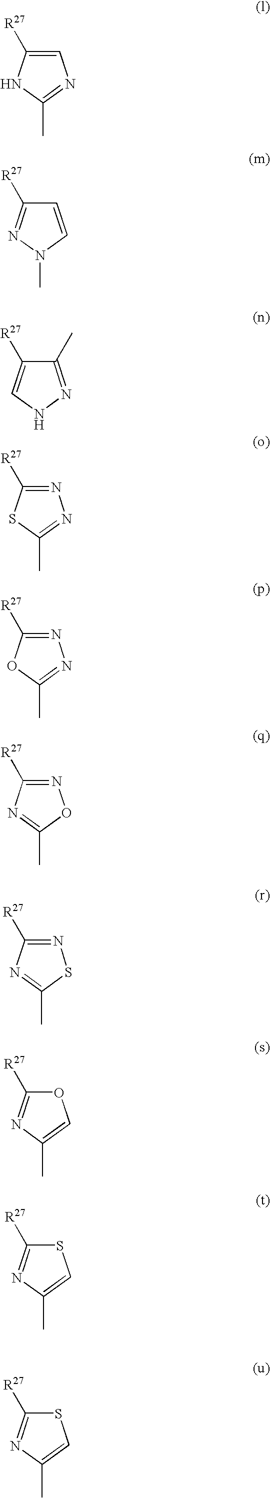

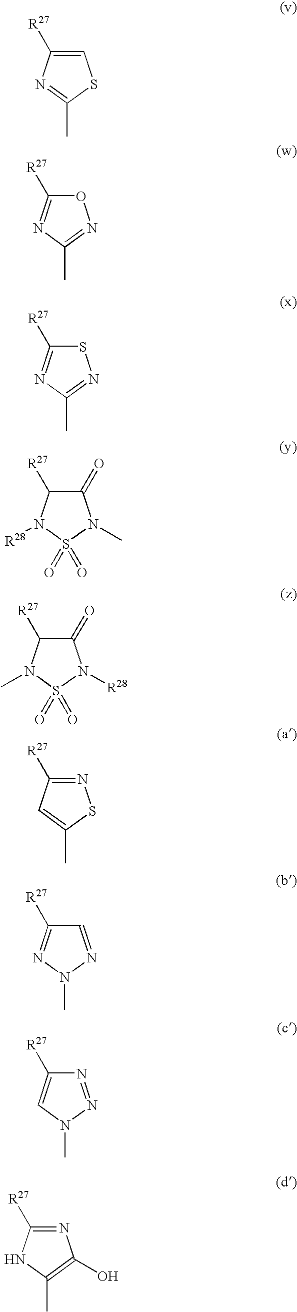

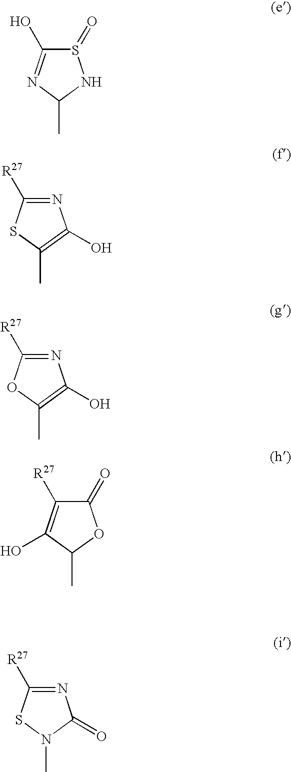

where p is 1 or 2, R²⁷ and R²⁸ are independently selected from hydrogen, hydroxy, (1-6C)alkoxy, thiol, (1-6C)alkylthio, —C(O)R²⁹, —S(O)R³⁰, —SO₂R³¹, —NR³²R³³, —NHCN, halogen and trihalomethyl, where R²⁹, R³⁰ and R³¹ are —OR³⁴, (1-6C)alkyl, —NR³²R³³ or trihalomethyl, R³² and R³³ are independently selected from hydrogen, (1-6C)alkyl, —SO₂R³⁴ and —COR³⁵, where R³⁵ is (1-6C)alkyl or trihalomethyl, and R³⁴ is hydrogen, (1-6C)alkyl or trihalomethyl and R¹³ is selected from hydrogen, (1-6C)alkyl, hydroxy, halo, amino, cyano, ((1-3C)alkyl)CONH—, carboxy, (1-6C)alkoxy, (1-6C)alkoxycarbonyl, carbamoyl, N-((1-6C)alkyl)carbamoyl, halo((1-6C)alkyl) (such as trifluoromethyl), (1-6C)alkylsulphonyl or (1-6C)alkylsulphinyl. Particular examples of R²⁷ or R²⁸ are hydroxy.

Further particular examples of carboxylic acid mimic or bioisostere groups include

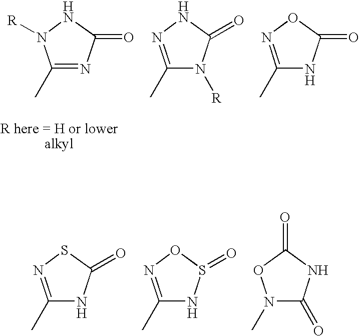

Particular carboxylic acid mimic or bioisosteres are a tetrazole group of sub-formula (b) and —C(O)NHS(O)₂Me.

In this specification the term “alkyl” includes both straight and branched chain alkyl groups, unless otherwise stated, and references to individual alkyl groups such as “propyl” are specific for the straight chain version only. An analogous convention applies to other generic terms. Unless otherwise stated the term “alkyl” advantageously refers to chains with 1-10 carbon atoms, suitably from 1-6 carbon atoms, preferably 1-4 carbon atoms.

In this specification the term “alkoxy” means an alkyl group as defined hereinbefore linked to an oxygen atom.

Particular values include for linear (1-3C)alkyl, methyl, ethyl and propyl; for (1-4C)alkyl, methyl, ethyl, propyl and butyl; for (2-3C)alkenyl, ethenyl; for (2-3C)alkynyl, ethynyl; for (1-2C)alkoxy, methoxy and ethoxy; for (1-6C)alkoxy and (1-4C)alkoxy, methoxy, ethoxy and propoxy.

Particular values include for any carbon atom in a linear (1-3C)alkyl, (1-2C)alkoxy, (1-4C)alkyl or (1-4C)alkoxy group that may be optionally substituted by up to 3 fluoro atoms, a group such as, for example, trifluoromethyl, difluoromethyl, difluoromethoxy or trifluoromethoxy.

For the avoidance of doubt it is to be understood that where in this specification a group is qualified by ‘hereinbefore defined’ or ‘defined hereinbefore’ the said group encompasses the first occurring and broadest definition as well as each and all of the particular definitions for that group.

If not stated elsewhere, suitable optional substituents for a particular group are those as stated for similar groups herein.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as Group (I) (alkali metal) salt, Group (II) (alkaline earth) metal salt, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions.

Suitable pharmaceutically-acceptable salts also include those mentioned in, for example, Berge et al. (J. Pharm. Sci., 1977, 66, 1-19) and/or Handbook of Pharmaceutical Salts: Properties, Selection and Use by Stahl and Wermuth (Wiley-VCH, 2002).

However, to facilitate isolation of the salt during preparation, salts which are less soluble in the chosen solvent may be preferred whether pharmaceutically-acceptable or not.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DGAT1 activity and is not to be limited merely to any one tautomeric form utilised within the formulae drawings. Also included are isotopes of certain atoms, for example, a deuterium atom in place of a hydrogen atom.

Also provided are pro-drugs of the compounds of formula (I), or a pharmaceutically-acceptable salt thereof.

Pro-drugs of compounds of formula (I), and salts thereof, are also within the scope of the invention.

Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and

### H. Bundgaard, Chapter 5 “Design and Application of Prodrugs”, by H. Bundgaard p. 113-191 (1991);

### c) H. Bundgaard, Advanced Drug Delivery Reviews, 8, 1-38 (1992);

### d) H. Bundgaard, et al., Journal of Pharmaceutical Sciences, 77, 285 (1988); and

### e) N. Kakeya, et al., Chem Pharm Bull, 32, 692 (1984).

Examples of such prodrugs are in vivo cleavable esters of a compound of the invention. An in vivo cleavable ester of a compound of the invention containing a carboxy group is, for example, a pharmaceutically-acceptable ester which is cleaved in the human or animal body to produce the parent acid. Suitable pharmaceutically-acceptable esters for carboxy include (1-6C)alkyl esters, for example methyl or ethyl; (1-6C)alkoxymethyl esters, for example methoxymethyl; (1-6C)alkanoyloxymethyl esters, for example pivaloyloxymethyl; phthalidyl esters; (3-8C)cycloalkoxycarbonyloxy(1-6C)alkyl esters, for example 1-cyclohexylcarbonyloxyethyl; 1,3-dioxolan-2-ylmethyl esters, for example 5-methyl-1,3-dioxolan-2-ylmethyl; (1-6C)alkoxycarbonyloxyethyl esters, for example 1-methoxycarbonyloxyethyl; aminocarbonylmethyl esters and mono- or di-N-((1-6C)alkyl) versions thereof, for example N,N-dimethylaminocarbonylmethyl esters and N-ethylaminocarbonylmethyl esters; and may be formed at any carboxy group in the compounds of this invention. An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include (1-6C)alkanoyl esters, for example acetyl esters; and benzoyl esters wherein the phenyl group may be substituted with aminomethyl or N-substituted mono- or di-(1-6C)alkyl aminomethyl, for example 4-aminomethylbenzoyl esters and 4-N,N-dimethylaminomethylbenzoyl esters.

Particular prodrugs are (1-4C)alkyl esters of the carboxyclic acid in compounds of formula (I).

It will be appreciated by those skilled in the art that certain compounds of formula (I) contain asymmetrically substituted carbon and/or sulfur atoms, and accordingly may exist in, and be isolated in, optically-active and racemic forms. Some compounds of formula (I) may exhibit polymorphism. It is to be understood that the present invention encompasses any racemic, optically-active, polymorphic or stereoisomeric form, or mixtures thereof, which form possesses properties useful in the inhibition of DGAT1 activity, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DGAT1 activity by the standard tests described hereinafter.

It is also to be understood that certain compounds of the formula (I) and salts thereof can exist in solvated as well as unsolvated forms such as, for example, hydrated forms. It is to be understood that the invention encompasses all such solvated forms which inhibit DGAT1 activity.

As stated before, we have discovered a range of compounds that have good DGAT1 inhibitory activity. They have good physical and/or pharmacokinetic properties in general. The following compounds possess in particular, desirable properties, such as, for example, pharmaco-kinetic/dynamic and/or toxicological profiles and/or selective activity for DGAT1.

In one aspect, it will be appreciated that in compounds of formula (I) the phenyl ring bearing the defined fluoro group and the defined Z (e.g. carboxy) group (or suitable replacement thereof) are in either a cis- or a trans-arrangement across the cyclohexyl ring, in relation to each other. Where appropriate the invention encompasses both the cis- and trans-isomers. Techniques for separation of such isomers are well known in the art.

Thus, in one aspect, the phenyl ring bearing the defined fluoro group and the defined carboxy group are in a cis-configuration across the cyclohexyl ring, to give a compound of formula (IA), wherein the variables are as defined hereinbefore or hereinafter:

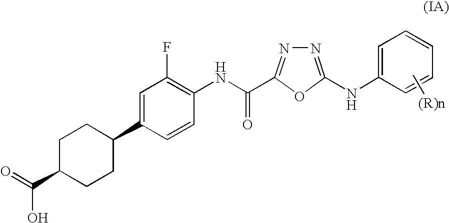

Thus, in another aspect, the phenyl ring bearing the defined fluoro group and the defined carboxy group are in a trans-configuration across the cyclohexyl ring, to give a compound of formula (IB), wherein the variables are as defined hereinbefore or hereinafter:

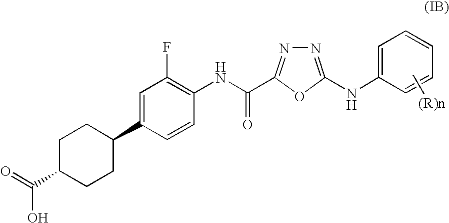

References hereinbefore or hereinafter to a compound of formula (I) are taken to apply also to compounds of formulae (IA) and (IB).

In one embodiment of the invention there are provided compounds of formulae (I), (IA) and (IB), in an alternative embodiment there are provided salts, particularly pharmaceutically-acceptable salts of compounds of formulae (I), (IA) and (IB). In a further embodiment, there are provided pro-drugs, particularly in-vivo cleavable esters, of compounds of formulae (I), (IA) and (IB). In a further embodiment, there are provided salts, particularly pharmaceutically-acceptable salts of pro-drugs of compounds of formulae (I), (IA) and (IB).

Particular values of substituents in compounds of formulae (I), (IA) and (IB) are as follows (such values may be used where appropriate with any of the other values, definitions, claims or embodiments defined hereinbefore or hereinafter).

(a) n is 1, 2 or 3;

(b) n is 2 or 3;

(c) n is 2;

(d) n is 3;

(e) R is fluoro or chloro;

(f) R is fluoro;

(g) n is 2 or 3 and R is fluoro

(h) n is 1 and R is trifloromethyl;

(i) n is 1 and R is difloromethoxy;

(j) n is 1 and R is trifloromethoxy.

(k) Z is carboxy.

In one embodiment the present invention provides a compound of formula (IC) below, or a pharmaceutically-acceptable salt thereof, wherein n is 2 or 3 and R is independently selected from fluoro, chloro, trifluoromethyl, difluoromethoxy and trifluoromethoxy.

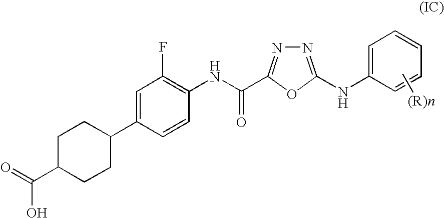

In another embodiment the present invention provides a compound of formula (I) or (IC), wherein n is 2 or 3 and R is independently selected from fluoro, chloro, trifluoromethyl, difluoromethoxy and trifluoromethoxy.

In another embodiment the present invention provides a compound of formula (I) or (IC), or a pharmaceutically-acceptable salt thereof, wherein n is 2 or 3 and R is fluoro.

In another embodiment the present invention provides a compound of formula (I), wherein n is 2 or 3 and R is fluoro.

In another embodiment the present invention provides a compound of formula (IB), or a pharmaceutically-acceptable salt thereof,

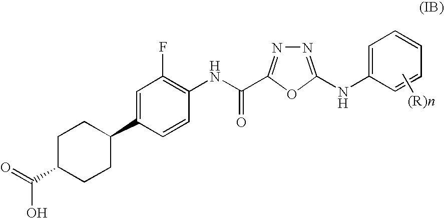

wherein n is 2 or 3 and R is fluoro.

In another embodiment the present invention provides a compound of formula (IB),

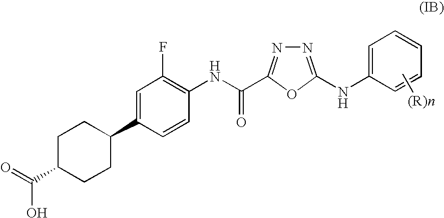

wherein n is 2 or 3 and R is fluoro.

Further particular compounds of the invention are each of the Examples, each of which provides a further independent aspect of the invention. In further aspects, the present invention also comprises any particular compounds of the Examples or a pharmaceutically-acceptable salt thereof (such as, for example, a sodium, magnesium, tert-butylammonium, tris(hydroxymethyl)methylammonium, triethanolammonium, diethanolammonium, ethanolammonium, methylethanolammonium, diethylammonium or nicotinamide salt).

A compound of formula (I) and its salts may be prepared by any process known to be applicable to the preparation of chemically related compounds. Such processes, when used to prepare a compound of the formula (I), or a pharmaceutically-acceptable salt thereof, are provided as a further feature of the invention.

In a further aspect the present invention also provides that the compounds of the formula (I) and salts thereof, can be prepared by the following processes, the processes of the Examples and analogous processes (wherein all variables are as hereinbefore defined for a compound of formula (I) unless otherwise stated) and thereafter if necessary any protecting groups can be removed and/or an appropriate salt formed. Any defined carboxylic acid groups may be replaced as appropriate by a mimic or bioisostere thereof.

Also included as an aspect of the invention are the compounds obtainable by any of the processes described herein.

A compound of formula (I) and its pharmaceutically-acceptable salts may be prepared by any process known to be applicable to the preparation of chemically related compounds. Such processes, when used to prepare a compound of the formula (I), or a pharmaceutically-acceptable salt thereof, are provided as a further feature of the invention.

In a further aspect the present invention also provides that the compounds of the formula (I) and pharmaceutically-acceptable salts or prodrugs thereof, can be prepared by a process a) to c) as follows (wherein all variables are as hereinbefore defined for a compound of formula (I) unless otherwise stated, and wherein cis- or trans-compounds can be prepared by use of appropriate intermediate compounds and compounds with different Z groups may be prepared by use of appropriate compounds):

a) reaction of a compound of formula (I) to form another compound of formula (I);

b) reaction of an amine-ester of formula (2) with a carboxylate salt of formula (3);

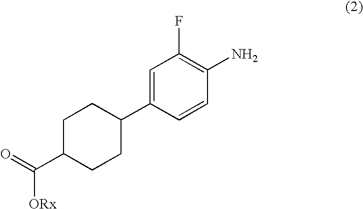

wherein Rx is, for example, methyl, ethyl or t-butyl

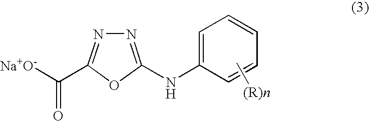

followed by deprotection (using base hydrolysis when Rx=methyl or ethyl) and acid deprotection when Rx=t-butyl).

c) cyclisation of a compound of formula (4)

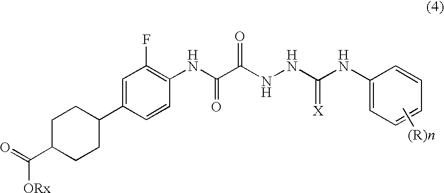

where X=S or O;

wherein Rx is, for example, methyl, ethyl or t-butyl

and thereafter if necessary, removing any protecting groups, and/or forming a pharmaceutically-acceptable salt or prodrug thereof.

In the structures & schemes herein, R and n are as defined above.

### Process a)

Examples of conversions of a compound of formula (I) into another compound of Formula (I), well known to those skilled in the art, include functional group interconversions such as hydrolysis (in particular ester hydrolysis), oxidation or reduction (such as the reduction of an acid to an alcohol) or conversion of an acid to an amide, and/or further functionalisation by standard reactions.

### Process b)

Compounds of formula (2) may be made by application of standard synthetic methods well known in the art, such as illustrated below in Scheme 1 and in the accompanying Examples.

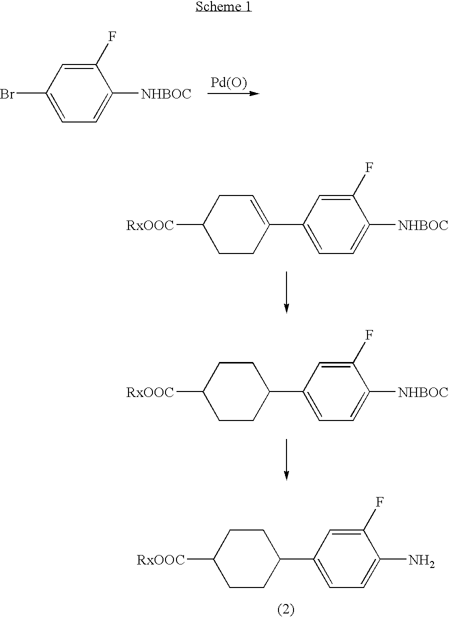

Compounds of formula (2) which may have chiral centres or exist in different isomeric forms such as cis/trans isomers, may be prepared as individual isomers as appropriate.

In Scheme 1 the appropriate stereochemistry may also be obtained by separation of the desired isomer by standard procedures such as chromatography or recrystallisation. Chromatography or recrystallisation may be performed on either of the last two compounds shown in Scheme 1. The compound of formula (2) may be prepared as a salt (such as the hydrochloride salt) to aid recrystallisation.

In Scheme 1 the Rx group may be removed by hydrolysis, for example using KOH.

Compounds of formula (3) may be made by alkaline hydrolysis of ester (3a) as prepared using a published procedure (J. Het. Chem. 1977, 14, 1385-1388). Ester (3a) may be made by cyclisation of a compound of formula (3b) in a similar manner as described in process c) for compounds of formula (4).

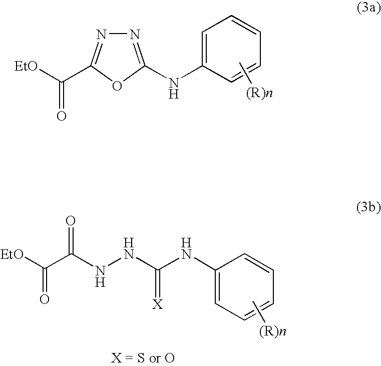

An alternative method for making compounds of formula (3a) is illustrated below:

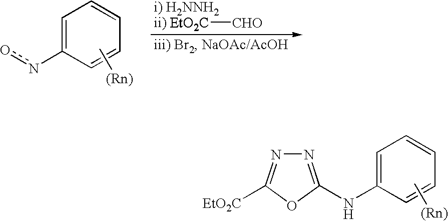

Compounds of formula (2) may be coupled with compounds of formula (3) under standard conditions for formation of amide bonds. For example using an appropriate coupling reaction, such as a carbodiimide coupling reaction performed with EDAC, optionally in the presence of DMAP, in a suitable solvent such as DCM, chloroform or DMF at room temperature.

### Process c)

Compounds of formula (4) and (3b) where X is S may be made by reaction of an aminocarbonyl acylhydrazine or ethoxycarbonyl acylhydrazine with a thioisocyanate or thioisocyanate equivalent such as aminothiocarbonylimidazole in a suitable solvent such as DMF or MeCN at a temperature between 0 and 100° C. The preparation of aminocarbonyl acylhydrazines from anilines and of ethoxycarbonyl acylhydrazines is well known in the art. For example reaction of an aniline with methyl chlorooxoacetate in the presence of pyridine in a suitable solvent such as DCM followed by reaction with hydrazine in a suitable solvent such as ethanol at a temperature between 0 and 100° C.

The compound of formula (4) may then be cyclised using, for example agents such as carbonyldiimidazole, or tosyl chloride and a suitable base (such as triethylamine), under conditions known in the art.

Compounds of formula (4) and (3b) where X is O may be made by analogous use of the appropriate isocyanate.

An example of process c) is shown in Scheme 2:

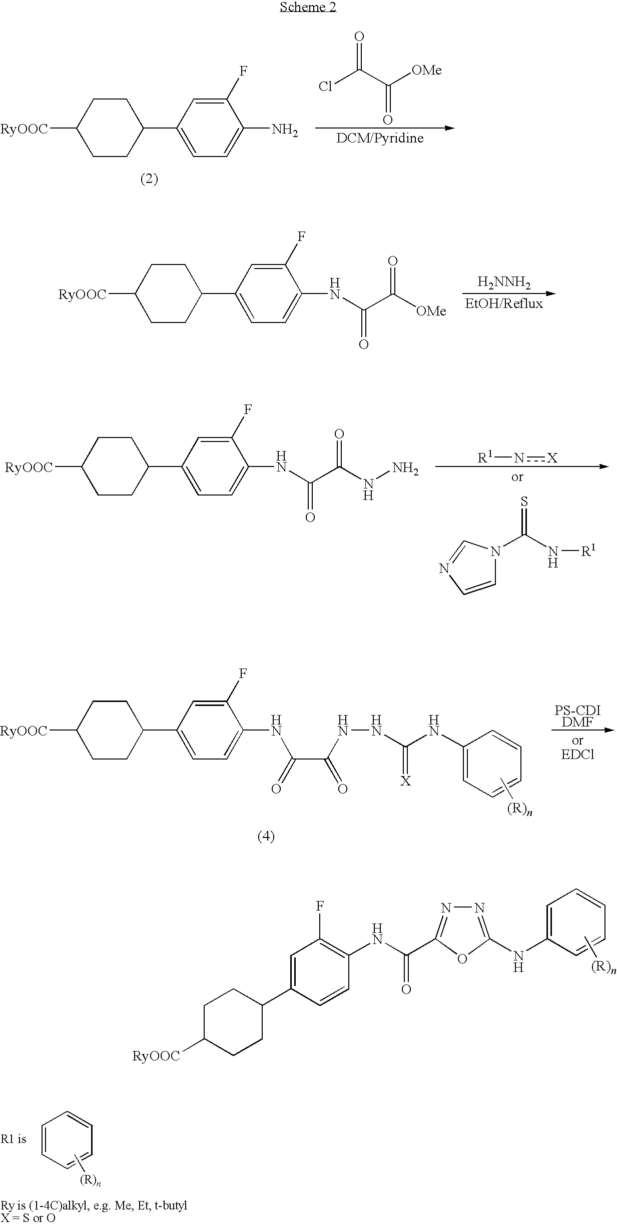

Compounds in Scheme 2 which may have chiral centres or exist in different isomeric forms such as cis/trans isomers, may be prepared as individual isomers as appropriate.

In Scheme 2 the Ry group may be removed by appropriate deprotection techniques.

Iso(thio)cyanates R¹—NCX (where X is O or S) are commercially available or may be made by reaction of the acid chlorides R¹—NH₂ with for example (thio)phosgene or a (thio)phosgene equivalent followed by a suitable base (such as triethylamine).

Compounds of formula (4) may be made from compounds of formula (2).

It will be appreciated that certain of the various ring substituents R in the compounds of the present invention, may be introduced by standard aromatic substitution reactions or generated by conventional functional group modifications either prior to or immediately following the processes mentioned above, and as such are included in the process aspect of the invention. Such reactions may convert one compound of the formula (I) into another compound of the formula (I), for example interconversion of one Z group to another Z group. The reagents and reaction conditions for such procedures are well known in the chemical art.

If not commercially available, the necessary starting materials for the procedures such as those described above may be made by procedures which are selected from standard organic chemical techniques, techniques which are analogous to the synthesis of known, structurally similar compounds, techniques which are described or illustrated in the references given above, or techniques which are analogous to the above described procedure or the procedures described in the examples. The reader is further referred to Advanced Organic Chemistry, 5^{th} Edition, by Jerry March and Michael Smith, published by John Wiley & Sons 2001, for general guidance on reaction conditions and reagents.

It will be appreciated that some intermediates to compounds of the formula (I) are also novel and these are provided as separate independent aspects of the invention.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl or SEM may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art, or they may be removed during a later reaction step or work-up.

The skilled organic chemist will be able to use and adapt the information contained and referenced within the above references, and accompanying Examples therein and also the examples herein, to obtain necessary starting materials, and products.

The removal of any protecting groups and the formation of a pharmaceutically-acceptable salt are within the skill of an ordinary organic chemist using standard techniques. Furthermore, details on the these steps has been provided hereinbefore.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further aspect of the invention there is provided a pharmaceutical composition which comprises a compound of formula (I), (IA) or (IB) or (IC) as defined hereinbefore or a pharmaceutically-acceptable salt thereof, in association with a pharmaceutically-acceptable excipient or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

Compounds of the invention, for example the Examples described herein, can be formulated as a nanosuspension (typically with mean particle size of <1 μm), for example, in a vehicle of polyvinylpyrrolidone/Aerosol OT, for example as follows:

Typical vehicle preparation (e.g 100 ml):

Weigh 0.67 gram polyvinylpyrrolidone (Kollidon 25 grade ex. BASF) and 0.033 gram Aerosol OT-100 (sodium dioctyl sulphosuccinate ex. Cyclex Industries) into a 100 ml volumetric flask. Add approximately 70 ml of de-ionised water and sonicate the flask until a solution is formed. Make up to volume with de-ionised water to produce a solution containing Polyvinylpyrrolidone (0.67% w/v)/Aerosol OT (0.033% w/v) in de-ionised water.

Nanosuspension Preparation:

The required amount of compound to produce the final concentration of drug in suspension is weighed into a suitable pre-volume marked vessel.

A small amount of vehicle is added to the compound and mixed to wet the compound and form a slurry. The slurry is then made up to volume with vehicle.

The so-formed slurry is then bead milled in a zirconia milling pot containing 0.6-0 8 mm diameter zirconia milling beads on a Fritsch P7 planetary micromill rotating at 800 rpm. Milling times are usually 4×30 minutes milling runs with a 15 minute cooling period between each run. After milling the milling pot is allowed to cool to room temperature and the nanosuspension separated from the beads.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

According to a further aspect of the present invention there is provided a compound of formula (I), (IA) and/or (IB), or a pharmaceutically acceptable salt, or a pro-drug thereof as defined hereinbefore for use in a method of treatment of the human or animal body by therapy.

We have found that compounds of the present invention inhibit DGAT1 activity and are therefore of interest for their blood glucose-lowering effects.

A further feature of the present invention is a compound of formula (I), (IA) and/or (IB), or a pharmaceutically-acceptable salt, or a pro-drug thereof for use as a medicament.

Conveniently this is a compound of formula (I), (IA) and/or (IB), or a pharmaceutically-acceptable salt, or a pro-drug thereof, for (use as a medicament for) producing an inhibition of DGAT1 activity in a warm-blooded animal such as a human being.

Particularly this is a compound of formula (I), (IA) and/or (IB), or a pharmaceutically-acceptable salt, or a pro-drug thereof, for (use as a medicament for) treating diabetes mellitus and/or obesity in a warm-blooded animal such as a human being.

Thus according to a further aspect of the invention there is provided the use of a compound of formula (I), (IA) and/or (IB), or a pharmaceutically-acceptable salt, or a pro-drug thereof in the manufacture of a medicament for use in the production of an inhibition of DGAT1 activity in a warm-blooded animal such as a human being.

Thus according to a further aspect of the invention there is provided the use of a compound of formula (I), (IA) and/or (IB), or a pharmaceutically-acceptable salt, or a pro-drug thereof in the manufacture of a medicament for use in the treatment of diabetes mellitus and/or obesity in a warm-blooded animal such as a human being.

According to a further aspect of the invention there is provided a pharmaceutical composition which comprises a compound of formula (I), (IA) and/or (IB) as defined hereinbefore, or a pharmaceutically-acceptable salt, or a pro-drug thereof, in association with a pharmaceutically-acceptable excipient or carrier for use in producing an inhibition of DGAT1 activity in an warm-blooded animal, such as a human being.

According to a further aspect of the invention there is provided a pharmaceutical composition which comprises a compound of formula (I), (IA) and/or (IB) as defined hereinbefore, or a pharmaceutically-acceptable salt, or a pro-drug thereof, in association with a pharmaceutically-acceptable excipient or carrier for use in the treatment of diabetes mellitus and/or obesity in an warm-blooded animal, such as a human being.

According to a further feature of the invention there is provided a method for producing an inhibition of DGAT1 activity in a warm-blooded animal, such as a human being, in need of such treatment which comprises administering to said animal an effective amount of a compound of formula (I), (IA) and/or (IB), or a pharmaceutically-acceptable salt, or a pro-drug thereof as defined hereinbefore.

According to a further feature of the invention there is provided a method of treating diabetes mellitus and/or obesity in a warm-blooded animal, such as a human being, in need of such treatment which comprises administering to said animal an effective amount of a compound of formula (I), (IA) and/or (IB), or a pharmaceutically-acceptable salt, or a pro-drug thereof as defined hereinbefore.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. In another embodiment a daily dose is in the range of 0.01-50 mg/kg, particularly 0.01-10 mg/kg, 0.01-1 mg/kg or 0.01-0.1 mg/kg. However the daily dose will necessarily be varied depending upon the host treated, the particular route of administration, and the severity of the illness being treated. Accordingly the optimum dosage may be determined by the practitioner who is treating any particular patient.

As stated above compounds defined in the present invention are of interest for their ability to inhibit the activity of DGAT1. A compound of the invention may therefore be useful for the prevention, delay or treatment of a range of disease states including diabetes mellitus, more specifically type 2 diabetes mellitus (T2DM) and complications arising there from (for example retinopathy, neuropathy and nephropathy), impaired glucose tolerance (IGT), conditions of impaired fasting glucose, metabolic acidosis, ketosis, dysmetabolic syndrome, arthritis, osteoporosis, obesity and obesity related disorders, (which include peripheral vascular disease, (including intermittent claudication), cardiac failure and certain cardiac myopathies, myocardial ischaemia, cerebral ischaemia and reperfusion, hyperlipidaemias, atherosclerosis, infertility and polycystic ovary syndrome); the compounds of the invention may also be useful for muscle weakness, diseases of the skin such as acne, various immunomodulatory diseases (such as psoriasis), HIV infection, inflammatory bowel syndrome and inflammatory bowel disease such as Crohn's disease and ulcerative colitis.

In particular, the compounds of the present invention are of interest for the prevention, delay or treatment of diabetes mellitus and/or obesity and/or obesity related disorders. In one aspect, the compounds of the invention are used for prevention, delay or treatment of diabetes mellitus. In another aspect, the compounds of the invention are used for prevention, delay or treatment of obesity. In a further aspect, the compounds of the invention are used for prevention, delay or treatment of obesity related disorders.

The inhibition of DGAT1 activity described herein may be applied as a sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets. For example such conjoint treatment may be beneficial in the treatment of metabolic syndrome [defined as abdominal obesity (as measured by waist circumference against ethnic and gender specific cut-points) plus any two of the following: hypertriglyceridemia (>150 mg/dl; 1.7 mmol/l); low HDLc (<40 mg/dl or <1.03 mmol/l for men and <50 mg/dl or 1.29 mmol/l for women) or on treatment for low HDL (high density lipoprotein); hypertension (SBP≧130 mmHg DBP≧85 mmHg) or on treatment for hypertension; and hyperglycemia (fasting plasma glucose≧100 mg/dl or 5.6 mmol/l or impaired glucose tolerance or pre-existing diabetes mellitus)-International Diabetes Federation & input from IAS/NCEP].

Such conjoint treatments may include the following main categories:

1) Anti-obesity therapies such as those that cause weight loss by effects on food intake, nutrient absorption or energy expenditure, such as orlistat, sibutramine and the like.
2) Insulin secretagogues including sulphonylureas (for example glibenclamide, glipizide), prandial glucose regulators (for example repaglinide, nateglinide);
3) Agents that improve incretin action (for example dipeptidyl peptidase IV inhibitors, and GLP-1 agonists);
4) Insulin sensitising agents including PPARgamma agonists (for example pioglitazone and rosiglitazone), and agents with combined PPARalpha and gamma activity;
5) Agents that modulate hepatic glucose balance (for example metformin, fructose 1, 6 bisphosphatase inhibitors, glycogen phopsphorylase inhibitors, glycogen synthase kinase inhibitors, glucokinase activators);
6) Agents designed to reduce the absorption of glucose from the intestine (for example acarbose);
7) Agents that prevent the reabsorption of glucose by the kidney (SGLT inhibitors);
8) Agents designed to treat the complications of prolonged hyperglycaemia (for example aldose reductase inhibitors);
9) Anti-dyslipidaemia agents such as, HMG-CoA reductase inhibitors (eg statins); PPAR α-agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
10) Antihypertensive agents such as β-blockers (eg atenolol, inderal); ACE inhibitors (eg lisinopril); Calcium antagonists (eg. nifedipine); Angiotensin receptor antagonists (eg candesartan), α-antagonists and diuretic agents (eg. furosemide, benzthiazide);
11) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin;
12) Agents which antagonise the actions of glucagon; and
13) Anti-inflammatory agents, such as non-steroidal anti-inflammatory drugs (eg. aspirin) and steroidal anti-inflammatory agents (eg. cortisone).

In addition to their use in therapeutic medicine, compounds of formula (I) and their pharmaceutically-acceptable salts are also useful as pharmacological tools in the development and standardisation of in vitro and in vivo test systems for the evaluation of the effects of inhibitors of DGAT1 activity in laboratory animals such as cats, dogs, rabbits, monkeys, rats and mice, as part of the search for new therapeutic agents.

In the above other pharmaceutical composition, process, method, use and medicament manufacture features, the alternative, particular and preferred embodiments of the compounds of the invention described herein also apply. The alternative, particular and preferred embodiments of the invention described herein also apply to a compound of formula (I), or a pharmaceutically-acceptable salt, or a pro-drug thereof.

As indicated above, all of the compounds, and their corresponding pharmaceutically-acceptable salts, are useful in inhibiting DGAT1. The ability of the compounds of formula (I), and their corresponding pharmaceutically-acceptable (acid addition) salts, to inhibit DGAT1 may be demonstrated employing the following enzyme assay:

### Human Enzyme Assay

See, for example, International Application WO 2005/044250.

The in vitro assay to identify DGAT1 inhibitors uses human DGAT1 expressed in insect cell membranes as the enzyme source (Proc. Natl. Acad. Sci. 1998, 95, 13018-13023). Briefly, sf9 cells were infected with recombinant baculovirus containing human DGAT1 coding sequences and harvested after 48 h. Cells were lysed by sonication and membranes isolated by centrifuging at 28000 rpm for 1 h at 4° C. on a 41% sucrose gradient. The membrane fraction at the interphase was collected, washed, and stored in liquid nitrogen.

DGAT1 activity was assayed by a modification of the method described by Coleman (Methods in Enzymology 1992, 209, 98-102). Compound at 0.0000256 μM-33 μM (final conc.) (typically 10 μM) was incubated with 4 μg/ml (final conc) membrane protein, 5 mM MgCl₂, and 100 μM 1,2 dioleoyl-sn-glycerol (dissolved in acetone with a final assay conc. of acetone of 10%) in a total assay volume of 200 μl in a 96 well plate. The reaction was started by adding ¹⁴C oleoyl coenzyme A (30 μM final concentration) and incubated at room temperature for 30 minutes. The reaction was stopped by adding 200 μl 2-propanol:heptane 7:1. Radioactive triolein product was separated into the organic phase by adding 300 μl heptane and 100 μl 0.1 M carbonate buffer pH 9.5. DGAT1 activity was quantified by counting aliquots of the upper heptane layer by liquid scintillography.

Using this assay the compounds generally show activity with an IC₅₀ below 10 μM, preferably below 10 μM (i.e. IC₅₀<10 μM), preferably <1 μM, more preferably <0.1 μM, particularly, <0.05 μM, and more particularly <0.01 μM. Stated figures are usually a mean of a number of measurements (usually 2 measurements) according to standard practice.

Examples 1 to 8 showed, respectively, an IC₅₀=0.016 μM; 0.028 μM; 0.011 μM; 0.012 μM; 0.0055 μM; 0.0037 μM; 0.0092 μM and 0.0047 μM.

The ability of the compounds of formula (I), and their corresponding pharmaceutically-acceptable (acid) salts, to inhibit DGAT1 may further be demonstrated employing the following whole cell assay.

### Measurement of Triglyceride Synthesis in HuTu 80 Cells

HuTu80 cells were cultured to confluency in 6 well plates in minimum essential media containing foetal calf serum. For the experiment, the medium was changed to serum-free medium and the cells pre-incubated with compound solubilised in DMSO (final concentration 0.1%) for 30 minutes. De novo lipogenesis was measured by the addition of 0.12 mM sodium oleate plus 1 μCi/mL ¹⁴C-sodium oleate complexed to 0.03 mM BSA to each well for a further 2 h. The cells were washed in phosphate buffered saline and solubilised in 1% sodium dodecyl sulfate. An aliquot was removed for protein determination using a protein estimation kit (Perbio) based on the method of Lowry (J. Biol. Chem., 1951, 193, 265-275). The lipids were extracted into the organic phase using a heptane:propan-2-ol:water (80:20:2) mixture followed by aliquots of water and heptane according to the method of Coleman (Methods in Enzymology, 1992, 209, 98-104). The organic phase was collected and the solvent evaporated under a stream of nitrogen. The extracts solubilised in iso-hexane:acetic acid (99:1) and lipids separated via normal phase high performance liquid chromatography (HPLC) using a Lichrospher diol-5, 4×250 mm column and a gradient solvent system of iso-hexane:acetic acid (99:1) and iso-hexane:propan-2-ol:acetic acid (85:15:1), flow rate of 1 mL/minute according to the method of Silversand and Haux (1997). Incorporation of radiolabel into the triglyceride fraction was analysed using a Radiomatic Flo-one Detector (Packard) connected to the HPLC machine.

### EXAMPLES

The following examples are for illustration purposes and are not intended to limit the scope of this application. Each exemplified compound represents a particular and independent aspect of the invention. In the following non-limiting Examples, unless otherwise stated:

(i) evaporations were carried out by rotary evaporation under reduced pressure and work-up procedures were carried out after removal of residual solids such as drying agents by filtration;
(ii) operations were carried out at room temperature, that is in the range 18-25° C. and generally under an atmosphere of an inert gas such as argon or nitrogen;
(iii) yields are given for illustration only and are not necessarily the maximum attainable;
(iv) the structures of the end-products of the Formula (I) were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;
(v) intermediates were not generally fully characterised and purity was assessed by thin layer chromatography (TLC), high-performance liquid chromatography (HPLC), infra-red (IR) or NMR analysis;
(vi) flash chromatography was carried out on silica unless otherwise stated with flash chromatography purifications run on Biotage SP1 or SP4 instruments using Biotage Silica columns;
(vii) mass spectra were recorded on a Finnigan LCQ Duo ion trap mass spectrometer equipped with an electrospray interface (LC-MS) or LC-MS system consisting of a Waters ZQ using a LC-Agilent 1100 LC system;
(viii) ¹H NMR measurements were performed on a Varian Mercury VXR 300 and 400 spectrometer, operating at a 1H frequency of 300 and 400 and Varian UNITY plus 400, 500 and 600 spectrometers, operating at 1H frequencies of 400, 500 and 600 respectively. Chemical shifts are given in ppm with the solvent as internal standard. Protons on heteroatoms such as NH and OH protons are only reported when detected in NMR and can therefore be missing.
(ix) HPLC separations were performed on a Waters YMC-ODS AQS-3 120 Angstrom 3×500 mm or on a Waters Delta Prep Systems using Kromasil C8, 10 μm columns. Acidic HPLC was carried out using gradients of mobilephase A: 100% ACN and mobilephase B: 5% ACN+95% H₂O+0.2% FA. Neutral HPLC was carried out using gradients of mobilephase A: 100% ACN and mobilephase B: 5% ACN+95% 0.1 M NH₄OAc.
(x) Reactions performed in a microwave oven were run in a Biotage Initiator Instrument.
(xi) Struc=Name/CambridgeSoft ELN has been used in the naming of compounds.
Other chemical nomenclature software packages, such as ACDName; ACDLabs Name: Release 9:00, product version 9.04 may be used.

The physical properties of compounds of the invention, such as the Examples described herein, can be measured by various techniques, such as those described below.

### X-Ray Powder Diffraction

X-ray powder diffraction spectra were determined by mounting a sample of the crystalline material on a Siemens single silicon crystal (SSC) wafer mount and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms. The collimated X-ray source was passed through an automatic variable divergence slit set at V20 and the reflected radiation directed through a 2 mm antiscatter slit and a 0.2 mm detector slit. The sample was exposed for 1 second per 0.02 degree 2-theta increment (continuous scan mode) over the range 2 degrees to 40 or 50 degrees 2-theta in theta-theta mode. The instrument was equipped with a scintillation counter as detector. Control and data capture was by means of a Dell Optiplex 686 NT 4.0 Workstation operating with Diffract+ software. Persons skilled in the art of X-ray powder diffraction will realise that the relative intensity of peaks can be affected by, for example, grains above 30 microns in size and non-unitary aspect ratios that may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values.

It is known that an X-ray powder diffraction pattern may be obtained which has one or more measurement errors depending on measurement conditions (such as equipment or machine used). In particular, it is generally known that intensities in an X-ray powder diffraction pattern may fluctuate depending on measurement conditions.

Persons skilled in the art of X-ray powder diffraction will realise that the relative intensity of peaks can be affected by, for example, grains above 30 microns in size and non-unitary aspect ratios, which may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values. (Jenkins, R & Snyder, R. L. ‘Introduction to X-Ray Powder Diffractometry’ John Wiley & Sons 1996; Bunn, C. W. (1948), Chemical Crystallography, Clarendon Press, London; Klug, H. P. & Alexander, L. E. (1974), X-Ray Diffraction Procedures).

Generally, a measurement error of a diffraction angle in an X-ray powder diffractogram is approximately plus or minus 0.5° 2-theta, and such a degree of a measurement error should be taken into account when considering the X-ray powder diffraction data. Furthermore, it should be understood that intensities might fluctuate depending on experimental conditions and sample preparation (preferred orientation).

The following definitions have been used.

| % Relative Intensity* | Definition |
|---|---|
| 25-100 | vs (very strong) |
| 10-25 | s (strong) |
| 3-10 | m (medium) |
| 1-3 | w (weak) |

*The relative intensities are derived from diffractograms measured with fixed slits Analytical Instrument: Siemens D5000.

When it is stated that the present invention relates to a crystalline form, the degree of crystallinity is conveniently greater than about 60%, more conveniently greater than about 80%, particularly greater than about 90% and more particularly greater than about 95%. Most particularly the degree of crystallinity is greater than about 98%.

### Differential Scanning Calorimetry (DSC)

Thermal events were analysed by differential scanning calorimetry on a TA DSC Q1000 or Q2000 instrument. Typically, less than 5 mg of material contained in a standard aluminium closed cup with pinhole over the temperature range 25° C. to 340° C. at a constant heating rate of 5° C. or 10° C. (e.g. using a TA DSC Q1000) per minute. A purge gas using nitrogen was used (flow rate 100 ml per minute).

### LIST OF ABBREVIATIONS WHICH MAY BE USED HEREIN

ACN Acetonitrile
aq aqueous
Boc tert-butyloxycarbonyl
Brine Saturated solution of sodium chloride in water
BSA Bovine Serum Albumine
DCE 1,2-dichloroethane
DCM Dichloromethane
DEE Diethylether
DIPEA N,N-Diisopropylethylamine
DMAP Dimethylaminopyridine
DMF N,N-dimethylformamide
DMSO Dimethylsulphoxide
Dppf 1,1′-bis(Diphenylphosphino)ferrocene
EDCI 1-Ethyl-3-(3-dimethylaminopropyl)carbodiimide hydrochloride
EDTA Ethylenediaminetetraacetic acid
EtOAc Ethyl acetate
EtOH Ethanol
FA Formic acid
HOAc Acetic acid
HPLC High-performance liquid chromatography
HWE Horner-Wadsworth-Emmons
Hz Hertz
IPA Isopropylalcohol
iPr isopropyl
LC Liquid chromatography
m-CPBA meta-chloroperoxybenzoicacid
MeOH Methanol
MHz Megahertz
mL Millilitre
MS Mass spectra
NMM N-methylmorpholine
NMP N-methylpiperazine
NMR Nuclear magnetic resonance
OAc acetate
Ph Phenyl
PyBOP Benzotriazol-1-yl-oxytri-pyrrolidinophosphonium hexafluorophosphate
PyBROP Bromo-tris-pyrrolidino-phosphonium hexafluorophosphate
RT Room temperature
sat saturated
TEA Triethylamine
Tf trifluoromethylsulfonyl
TFA Trifluoroacetic acid
THF Tetrahydrofurane
TLC Thin layer chromatography
Ts p-toluenesulfonyl.

### Example 1

### (1r,4r)-4-(3-Fluoro-4-(5-(4-(trifluoromethyl)phenylamino)-1,3,4-oxadiazole-2-carboxamido)phenyl)cyclohexanecarboxylic acid

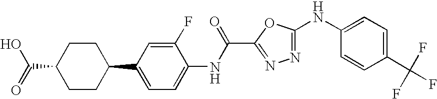

Sodium hydroxide (2M; 3.56 mL, 7.12 mmol) was added to Intermediate 1-1 (741 mg, 1.42 mmol) in MeOH (25 mL). The resulting solution was stirred for 16 hours.
The reaction mixture was evaporated and the aqueous residue (20 mL) was adjusted to pH 2 with 2M HCl (5 mL). The suspension was filtered and dried to afford the crude product. The crude product was purified by crystallisation from EtOH to afford the title compound (37.0 mg, 5.28%) as a white crystalline solid.

¹H NMR (400.13 MHz, DMSO-d₆) δ 1.38-1.55 (4H, m), 1.78-1.90 (2H, m), 1.90-2.05 (2H, m), 2.20-2.30 (1H, m), 2.40-2.60 (1H, m), 7.10 (1H, dd), 7.19 (1H, dd), 7.45 (1H, t), 7.78 (4H, dd), 10.70 (1H, s), 11.43 (1H, s), 12.02 (1H, s) m/z (ES+) (M+H)+=493.39.

### Intermediate 1-1: (1r,4r)-Ethyl 4-(3-fluoro-4-(5-(4-(trifluoromethyl)-phenylamino)-1,3,4-oxadiazole-2-carboxamido)phenyl)cyclohexanecarboxylate

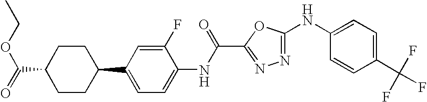

To a solution of Intermediate 1-2 (500 mg, 1.42 mmol) in DMF (10 mL) was added 4-(trifluoromethyl)phenyl isothiocyanate (347 mg, 1.71 mmol). The resulting mixture was stirred at 45° C. for 45 minutes, 1-(3-dimethylaminopropyl)-3-ethylcarbodiimide hydrochloride (396 mg, 2.06 mmol) was added and the mixture was stirred at 85° C. for 45 minutes. The reaction mixture was allowed to cool to ambient temperature, water (15 mL) was added and the precipitate was collected by filtration, washed with water (10 mL) and air dried to afford the title compound as a yellow solid, which was used without further purification. m/z (ESI+)(M+H)+=521.46.

### Intermediate 1-2: (1r,4r)-Ethyl 4-(3-fluoro-4-(2-hydrazinyl-2-oxoacetamido)-phenyl)cyclohexanecarboxylate

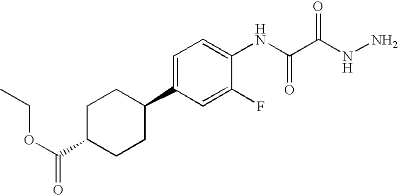

To a suspension of Intermediate 1-3 (3.55 g, 10.10 mmol) in EtOH (300 mL) was added hydrazine monohydrate (0.539 mL, 11.11 mmol). The resulting suspension was stirred at ambient temperature for 70 minutes. The reaction mixture was filtered and dried under vacuum to give the desired product as a white solid. The filtrate was evaporated to dryness to afford further product, which was combined with the filtered solid to provide the title compound (3.01 g, 85%) as a white solid.

¹H NMR (400 MHz, DMSO) δ 1.18 (3H, t), 1.40-1.53 (4H, m), 1.79-1.84 (2H, m), 1.97-2.02 (2H, m), 2.31-2.37 (1H, m), 2.51-2.55 (1H, m), 4.06 (2H, q), 4.61 (2H, s), 7.06-7.08 (1H, m), 7.14-7.18 (1H, m), 7.57 (1H, t), 10.07 (1H, s), 10.29 (1H, s); m/z (M+H)⁺352.

### Alternative Preparation of Intermediate 1-2

Hydrazine monohydrate (9.42 mL) was added to Intermediate 1-3 (62 g), in ethanol (930 mL) at 20° C. under nitrogen. The resulting thick slurry was stirred at 20° C. for 2 hours and the slurry was filtered, washed with ethanol and dried in the vac oven at 45° C. overnight to obtain (1r,4r)-ethyl 4-(3-fluoro-4-(2-hydrazinyl-2-oxoac etamido)phenyl)-cyclohexanecarboxylate (55.0 g, 89%) as a white solid.

¹H NMR (400 MHz, DMSO) d 1.19 (3H, t), 1.39-1.58 (4H, m), 1.84 (2H, d), 1.99 (2H, d), 2.30-2.40 (1H, m), 2.51-2.60 (1H, m), 4.07 (2H, q), 4.65 (2H, s), 7.08 (1H, dd), 7.18 (1H, dd), 7.56 (1H, t), 10.15 (1H, s), 10.35 (1H, s).

### Intermediate 1-3: (1r,4r)-Ethyl 4-(3-fluoro-4-(2-methoxy-2-oxoacetamido)-phenyl)cyclohexanecarboxylate

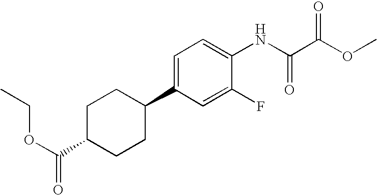

Methyl chlorooxoacetate (1.208 mL, 13.14 mmol) was added to a stirred solution of Intermediate 1-4 (3.05 g, 10.11 mmol) and pyridine (1.796 mL, 22.23 mmol) in DCM (80 mL). The resulting solution was stirred at ambient temperature for 90 minutes. The reaction mixture was diluted with DCM (50 mL), and washed with saturated brine (50 mL). The organic layer was dried over MgSO₄, filtered and evaporated to afford crude product which was purified by flash silica chromatography, elution gradient 0 to 20 to 60% EtOAc in isohexane. Pure fractions were evaporated to dryness to afford the title compound (3.55 g, 100%) as a white solid.

¹H NMR (400 MHz, CDCl₃) δ 1.27 (3H, t), 1.38-1.48 (2H, m), 1.54-1.64 (2H, m), 1.95-1.99 (2H, m), 2.09-2.14 (2H, m), 2.29-2.37 (1H, m), 2.47-2.55 (1H, m), 3.98 (3H, s), 4.14 (2H, q), 6.97-7.03 (2H, m), 8.24 (1H, t), 9.00 (1H, s); m/z (M+H)⁺352.

### Alternative Preparation of Intermediate 1-3

Methyl chlorooxoacetate (22.08 mL) was added drop wise to Intermediate 1-4 (53.5 g) and pyridine (31.5 mL) in DCM (802 mL) at 20° C. over a period of 10 minutes under nitrogen. The resulting solution was stirred at 20° C. for 2 hours. The reaction mixture was diluted with dichloromethane (535 mL) then washed sequentially with water (535 mL) and saturated brine (535 mL). The organic layer was dried over MgSO₄, filtered and evaporated to afford crude (1r,4r)-ethyl 4-(3-fluoro-4-(2-methoxy-2-oxoacetamido)phenyl)cyclohexanecarboxylate that was used directly in the next stage.

¹H NMR (400 MHz, CDCl₃) δ 1.25 (3H, t), 1.33-1.48 (2H, m), 1.48-1.64 (2H, m), 1.88-1.99 (2H, m), 2.03-2.14 (2H, m), 2.24-2.38 (1H, m), 2.43-2.56 (1H, m), 3.97 (3H, s), 4.13 (2H, q), 6.97 (2H, dd), 8.22 (1H, t), 9.00 (1H, s).

### Intermediate 1-4 (1r,4r)-Ethyl 4-(4-amino-3-fluorophenyl)cyclohexanecarboxylate hydrochloride

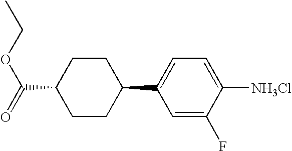

4M Hydrogen chloride in dioxane (17.81 mL, 71.23 mmol) was added to a stirred solution of Intermediate 1-5 (5.2 g, 14.23 mmol) in 1,4-dioxane (10 mL). The resulting solution was stirred at ambient temperature for 2 hours, the solution became solid over this time and then allowed to stand at ambient temperature overnight. The reaction mixture was evaporated, the crude solid was triturated with boiling EtOAc (˜25 mL) to give a solid which was collected by filtration and dried under vacuum to give the title compound (3.10 g) as a beige solid. A further crop of a solid (255 mg) came out from the filtrate and was filtered and dried then combined with the first crop to afford the title compound (3.36 g, 78%).

¹H NMR (400 MHz, DMSO) δ 1.18 (3H, t), 1.38-1.49 (4H, m), 1.79-1.81 (2H, m), 1.93-1.98 (2H, m), 2.28-2.36 (2H, m), 4.05 (2H, q), 6.97-6.99 (1H, m), 7.08-7.14 (2H, m); NH₂ not seen; m/z (M+H)⁺ 266.

### Alternative Preparation of Intermediate 1-4

4M Hydrogen chloride in dioxane (280 mL) was added to (1s,4s)-ethyl 4-(4-(tert-butoxycarbonylamino)-3-fluorophenyl)cyclohexanecarboxylate (82 g) in dioxane (820 mL) at 20° C. under nitrogen. The resulting solution was stirred at 20° C. for 5 days. The solvent was evaporated and the crude residue was triturated with EtOAc (820 mL) and the solid was collected by filtration and dried under vacum to give (1r,4r)-ethyl 4-(4-amino-3-fluorophenyl)cyclohexanecarboxylate hydrochloride (53.5 g, 79%) as a cream solid.

¹H NMR (400 MHz, DMSO) δ 1.18 (3H, t), 1.38-1.54 (4H, m), 1.75-1.86 (2H, m), 1.91-2.02 (2H, m), 2.26-2.40 (1H, m), 2.51-2.56 (1H, m), 4.06 (2H, q), 7.07 (1H, dd), 7.20 (1H, dd), 7.31 (1H, t).

### Intermediate 1-5: (1r,4r)-Ethyl 4-(4-(tert-butoxycarbonylamino)-3-fluorophenyl)cyclohexanecarboxylate (trans) and

### Intermediate 1-6: (1s,4s)-ethyl 4-(4-(tert-butoxycarbonylamino)-3-fluorophenyl)cyclohexanecarboxylate (cis)

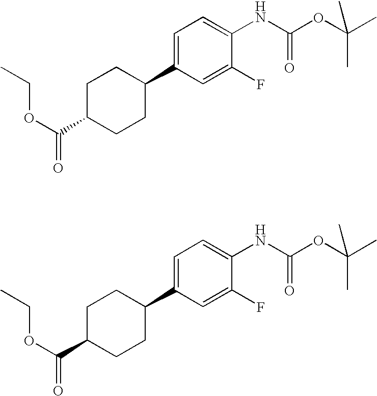

A mixture of Intermediate 1-7 (8.3 g, 22.84 mmol) and 10% palladium on carbon (1.215 g, 1.14 mmol) in EtOH (100 mL) was stirred under an atmosphere of hydrogen at ambient temperature for 5 hours. The reaction mixture was filtered to give solid A. The filtrate was concentrated and the residue recrystallised from EtOH (50 mL) to obtain the cis isomer (1.39 g). The mother liquor was concentrated by ˜50% and then stirred at ambient temperature with a few crystals of pure cis isomer for 90 minutes. The precipitate was collected by filtration and dried under vacuum to afford further cis compound as a white solid (131 mg). The filtrate was concentrated to leave a slightly turbid pale yellow gum (2.18 g) which was redissolved in abs EtOH (5 mL) and stirred with a small amt of pure cis compound at ambient temperature for 3 hrs and allowed to stand overnight. The precipitate was collected by filtration and dried under vacuum to afford pure cis compound. The filtrate was concentrated to afford the trans isomer. The filtered solid A was washed with DCM (2×25 mL) and the solvent evaporated to afford the pure cis isomer 3.02 g. Total recovery of the cis isomer=4.61 g, 12.62 mmol, 55.2% Total recovery of the trans isomer=2.04 g, 5.58 mmol, 24.4%

### (1s,4s)-ethyl 4-(4-(tert-butoxycarbonylamino)-3-fluorophenyl)cyclohexane-carboxylate

¹H NMR (400 MHz, CDCl₃) δ 1.28 (3H, t), 1.52 (9H, s), 1.57-1.67 (4H, m), 1.70-1.78 (2H, m), 2.21-2.28 (2H, m), 2.46-2.52 (1H, m), 2.66-2.69 (1H, m), 4.18 (2H, q), 6.58 (1H, s), 6.87-6.93 (2H, m), 7.91 (1H, t); m/z (M+Na)⁺ 388.

### (1r,4r)-ethyl 4-(4-(tert-butoxycarbonylamino)-3-fluorophenyl)cyclohexane-carboxylate

¹H NMR (400 MHz, CDCl₃) δ 1.18-1.32 (3H, m), 1.35-1.47 (2H, m), 1.52 (9H, s), 1.55-1.63 (2H, m), 1.92-1.97 (2H, m), 2.07-2.12 (2H, m), 2.27-2.35 (1H, m), 2.42-2.50 (1H, m), 4.10-4.17 (2H, m), 6.59 (1H, s), 6.87-6.91 (1H, m), 6.92-6.94 (1H, m), 7.93 (1H, t); m/z (M+Na)⁺ 388.

### Alternative Preparation of Intermediates 1-5 and 1-6

Intermediate 1-7 (200 g), platinum 5% wt on carbon (50% wet) JM type 128M (40 g) in ethanol (2000 mL) was stirred under an atmosphere of hydrogen at 5 bar and 40° C. for 4 hours. The reaction mixture was filtered and evaporated. The crude product was purified by crystallisation from EtOH (1800 mL) to afford (1s,4s)-ethyl 4-(4-(tert-butoxycarbonylamino)-3-fluorophenyl)cyclohexanecarboxylate (117 g, 54%) as a white solid. The mother liquors were evaporated and the material recrystallised from ethanol (150 mL) to afford (1s,4s)-ethyl 4-(4-(tert-butoxycarbonylamino)-3-fluorophenyl)cyclohexanecarboxylate (9.5 g, 5%). The mother liquors evaporated to dryness to provide a yellow oil (1r,4r)-ethyl 4-(4-(tert-butoxycarbonylamino)-3-fluorophenyl)cyclohexanecarboxylate (72.0 g, 35.8%).

¹H NMR (400 MHz, CDCl₃) δ 1.19 (3H, t), 1.27-1.58 (13H, m), 1.82-1.92 (2H, m), 1.95-2.08 (2H, m), 2.19-2.30 (1H, m), 2.33-2.50 (1H, m), 3.98-4.18 (2H, m), 6.55 (1H, s), 6.84 (2H, dd), 7.87 (1H, s).

### Cis/Trans Isomerisation and Deprotection Process to Give Intermediate 1-4

Potassium 2-methylpropan-2-olate (87 g) was added in one portion to Intermediate 1-6 (130.6 g) in tert-butanol (1310 mL) under nitrogen. After 4 hours saturated aqueous ammonium chloride (1300 mL) was added and the aqueous layer was pH adjusted to 1 using conc HCl and the aqueous layer extracted with ethyl acetate (2×650 mL). The organic layer was evaporated and taken up in ethyl acetate (1300 mL) and the combined organic layers were washed with 2M HCl (1300 mL) saturated aqueous brine (1300 mL) dried (MgSO₄) and evaporated to obtain crude trans isomer (136 g) which was dissolved in ethanol (1310 mL) and 4M hydrogen chloride in dioxane (448 mL) was added. The resulting yellow solution was stirred at 20° C. for 3 days, the solvent was evaporated and the crude residue was triturated with EtOAc (1310 mL) to give a solid which was collected by filtration washed with ethyl acetate and dried in the vac oven at 40° C. overnight to give (1r,4r)-ethyl 4-(4-amino-3-fluorophenyl)cyclohexanecarboxylate hydrochloride (99 g, 92%) as a white solid.

¹H NMR (400 MHz, DMSO) δ 1.18 (3H, t), 1.38-1.54 (4H, m), 1.75-1.86 (2H, m), 1.91-2.02 (2H, m), 2.26-2.40 (1H, m), 2.51-2.56 (1H, m), 4.06 (2H, q), 7.07 (1H, dd), 7.20 (1H, dd), 7.31 (1H, t).

### Intermediate 1-7: Ethyl 4-(4-(tert-butoxycarbonylamino)-3-fluorophenyl)cyclohex-3-enecarboxylate

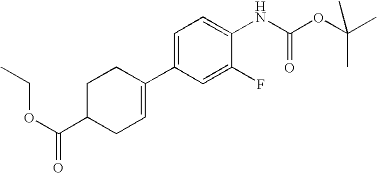

A solution of Intermediate 1-8 (6.85 g, 23.60 mmol) and Intermediate 1-9 (6.99 g, 23.60 mmol) in 1,2-dimethoxyethane (178 mL) and water (100 mL) was degassed by bubbling nitrogen through for 10 mins. 1,1′-Bis(diphenylphosphino)ferrocene-palladium dichloride (0.971 g, 1.18 mmol) and potassium carbonate (8.15 g, 59.00 mmol) were added and the reaction mixture was heated was stirred at 85° C. for 17 hours. The reaction mixture was allowed to cool to ambient temperature and then evaporated to remove the organic solvent. The residue was redissolved in EtOAc (200 mL) and washed with saturated brine (500 mL), the aqueous layer was re extracted with EtOAc (100 mL), organic extracts were combined, dried over MgSO₄, filtered and evaporated to afford crude product. The crude product was purified by flash silica chromatography, elution gradient 0 to 20% EtOAc in isohexane. Pure fractions were evaporated to dryness to afford ethyl 4-(4-(tert-butoxycarbonylamino)-3-fluorophenyl)cyclohex-3-enecarboxylate (86%) as a pale yellow gum.

¹H NMR (400 MHz, CDCl₃) δ 1.27 (3H, t), 1.52 (9H, s), 1.77-1.88 (1H, m), 2.14-2.20 (1H, m), 2.36-2.51 (4H, m), 2.55-2.63 (1H, m), 4.17 (2H, q), 6.05-6.08 (1H, m), 6.65 (1H, s), 7.06-7.13 (2H, m), 7.99 (1H, t); m/z (M-tBu)⁺ 308.

### Alternative Preparation of Intermediate 1-7

To Pd-118 [PdCl₂(dbpf)] (17.94 g) was added acetonitrile (769 mL) and the slurry was stirred for 5 mins then Potassium carbonate (152 g), water (769 mL) were charged followed by ethyl 4-(4,4,5,5-tetramethyl-1,3,2-dioxaborolan-2-yl)cyclohex-3-enecarboxylate (162 g) and after a further 5 mins tert-butyl 4-bromo-2-fluorophenylcarbamate (159 g) was charged and the reaction heated to 80° C. After 2 hours the reaction was cooled to ambient and the reaction mixture concentrated under reduced pressure and purified by flash silica chromatography, elution gradient 0 to 10% EtOAc in isohexane to afford ethyl 4-(4-(tert-butoxycarbonylamino)-3-fluorophenyl)cyclohex-3-enecarboxylate (167 g, 84%) as a yellow oil which solidified on standing.

¹H NMR (400.13 MHz, CDCl₃) δ 1.20 (3H, t), 1.45 (9H, s), 1.76 (1H, d), 2.08-2.12 (1H, m), 2.34-2.39 (4H, m), 2.50-2.53 (1H, m), 4.09 (2H, q), 6.00 (1H, s), 6.60 (1H, s), 6.98-7.02 (1H, m), 7.03-7.06 (1H, m), 7.92 (1H, s).

### Intermediate 1-8: tert-Butyl 4-bromo-2-fluorophenylcarbamate

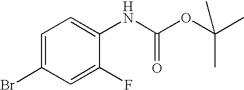

To an ice water cooled solution of 4-bromo-2-fluoroaniline (9.5 g, 50.00 mmol) in THF (50 mL) was added a 1M solution of sodium bis(trimethylsilyl)amide (100 mL, 99.99 mmol) in THF over a period of 30 minutes under nitrogen (internal temp 5° C.). The resulting deep blue solution was allowed to warm to ambient temperature over 10 minutes. A solution of di-tert-butyl dicarbonate (10.91 g, 50.00 mmol) in THF (50 mL) was added dropwise an the resulting mixture was stirred at ambient temperature for 90 minutes. The reaction mixture was poured onto saturated NaHCO₃ (600 mL) and extracted into ether (3×500 mL). The organic extracts were combined washed with saturated brine (500 mL), dried over MgSO₄, filtered and evaporated to afford crude product. The crude product was purified by flash silica chromatography, elution gradient 0 to 15% EtOAc in isohexane. Pure fractions were evaporated to dryness to afford tert-butyl 4-bromo-2-fluorophenylcarbamate (12.43 g, 86%) as a red solid.

¹H NMR (400 MHz, CDCl₃) δ 1.52 (9H, s), 6.66 (1H, s), 7.21-7.25 (2H, m), 8.01 (1H, t); m/z (ESI+) M+Na 313.

### Intermediate 1-9: Ethyl 4-(4,4,5,5-tetramethyl-1,3,2-dioxaborolan-2-yl)cyclohex-3-enecarboxylate

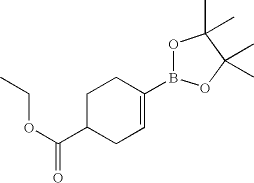

A solution of Intermediate 1-10 (16.7 g, 55.25 mmol) in dioxane (300 mL) was deoxygenated by bubbling nitrogen through for 15 minutes. Potassium acetate (16.27 g, 165.75 mmol), bis(pinacolato)diboron (15.43 g, 60.77 mmol), 1,1′-bis(diphenylphosphino)ferrocene (1.548 g, 2.76 mmol) and (1,1′-bis(diphenylphosphino)ferrocene)-dichloropalladium(II) (2.273 g, 2.76 mmol) were added and resulting suspension was stirred at 80° C. overnight. The reaction mixture was allowed to cool, evaporated to dryness and redissolved in EtOAc (300 mL), and washed with saturated brine (2×200 mL). The organic layer was dried over MgSO₄, filtered and evaporated to afford crude product. The crude product was purified by flash silica chromatography, elution gradient 0 to 20% EtOAc in isohexane. Pure fractions were evaporated to dryness to afford Ethyl 4-(4,4,5,5-tetramethyl-1,3,2-dioxaborolan-2-yl)cyclohex-3-enecarboxylate (6.99 g, 45.2%) as a colourless gum.

¹H NMR (400 MHz, CDCl₃) δ 1.25 (3H, t), 1.26 (12H, s), 1.58-1.65 (1H, m), 1.98-2.04 (1H, m), 2.07-2.18 (1H, m), 2.24-2.36 (3H, m), 2.47-2.56 (1H, m), 4.14 (2H, q), 6.53-6.55 (1H, m).

### Alternative Preparation of Intermediate 1-9

Ethyl 4-(trifluoromethylsulfonyloxy)cyclohex-3-enecarboxylate (325 g) was added as a solution in degassed dioxane (3250 mL) to bis(pinacolato)diboron (300 g), (1,1′-bis(diphenylphosphino)ferrocene)-dichloropalladium(II) acetone adduct (44.2 g) and 1,1′-bis(diphenylphosphino)ferrocene (30.1 g), Potassium acetate (317 g) in dioxane (2178 mL) at 20° C. under nitrogen. The red suspension was stirred at 80° C. for 1 hour. The solvent was evaporated. The crude product was taken up into ethyl acetate (4550 mL) and water (650 mL) washed with saturated aqueous brine (2×3250 mL) dried (MgSO₄) and evaporated. The crude brown oil was purified by flash silica chromatography, elution gradient 0 to 10% EtOAc in isohexane to afford ethyl 4-(4,4,5,5-tetramethyl-1,3,2-dioxaborolan-2-yl)cyclohex-3-enecarboxylate (190 g, 63.1%) as a yellow oil.

¹H NMR (400 MHz, CDCl₃) δ 1.16-1.22 (15H, m), 1.46-1.61 (1H, m), 1.88-1.99 (1H, m), 2.13-1.99 (1H, m), 2.15-2.31 (3H, m), 2.39-2.50 (1H, m), 4.02-4.11 (2H, m), 6.48 (1H, s).

### Intermediate 1-10: Ethyl 4-(trifluoromethylsulfonyloxy)cyclohex-3-enecarboxylate

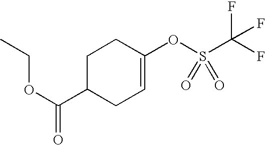

Trifluoromethanesulphonic anhydride (516 mL) was added drop wise to ethyl 4-oxocyclohexanecarboxylate (350 g), 2,6-lutidine (359 mL) in dichloromethane (3500 mL) at 20° C. over a period of 1 hour under nitrogen. The resulting solution was stirred at ambient temperature for 15 minutes; additional trifluoromethanesulphonic anhydride (172 mL) was charged drop wise after 1 hour additional trifluoromethanesulphonic anhydride (34 mL) was charged the red reaction mixture was evaporated to dryness and the crude product was purified by flash silica chromatography, elution gradient 0 to 10% EtOAc in isohexane to afford ethyl 4-(trifluoromethylsulfonyloxy)cyclohex-3-enecarboxylate (425 g, 68.4%) as a yellow oil.

¹H NMR (400.132 MHz, CDCl₃) δ 1.26 (3H, t), 1.87-1.98 (1H, m), 2.09-2.18 (1H, m), 2.38-2.49 (4H, m), 2.55-2.64 (1H, m), 4.16 (2H, q), 5.75-5.79 (1H, m)

### Example 2

### (1r,4r)-4-(4-(5-(4-(Difluoromethoxy)phenylamino)-1,3,4-oxadiazole-2-carboxamidol-3-fluorophenyl)cyclohexanecarboxylic acid

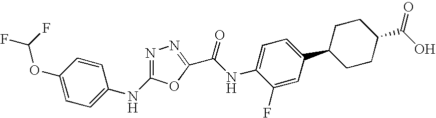

Sodium hydroxide (2M; 3.56 mL, 7.12 mmol) was added to Intermediate 2-1 (738 mg, 1.42 mmol) in MeOH (25 mL). The resulting solution was stirred for 16 hours. The reaction mixture was evaporated and the aqueous residue (20 mL) was adjusted to pH 2 with 2M HCl (5 mL). The suspension was filtered and dried to afford the crude product. The crude product was purified by crystallisation from AcOH to afford (1r,4r)-4-(4-(5-(4-(difluoromethoxy)phenylamino)-1,3,4-oxadiazole-2-carboxamido)-3-fluorophenyl)cyclohexanecarboxylic acid (320 mg, 45.8%) as a white crystalline solid.

¹H NMR (400.13 MHz, DMSO-d₆) δ 1.38-1.55 (4H, m), 1.78-1.90 (2H, m), 1.90-2.05 (2H, m), 2.20-2.30 (1H, m), 2.40-2.60 (1H, m), 7.06-7.25 (5H, m), 7.45 (1H, t), 7.62 (2H, dd), 10.63 (1H, s), 11.04 (1H, s), 12.02 (1H, s) m/z (ES+) (M+H)+=491.41.

### Intermediate 2-1: (1r,4r)-Ethyl 4-(4-(5-(4-(difluoromethoxy)phenylamino)-1-1,3,4-oxadiazole-2-carboxamidol-3-fluorophenyl)cyclohexanecarboxylate

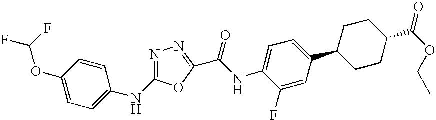

To a solution of Intermediate 1-2 (500 mg, 1.42 mmol) in DMF (20 mL) was added 1-(difluoromethoxy)-4-isothiocyanatobenzene (0.258 mL, 1.71 mmol). The resulting mixture was stirred at 45° C. for 45 minutes, 1-(3-dimethylaminopropyl)-3-ethylcarbodiimide hydrochloride (396 mg, 2.06 mmol) was added and the mixture was stirred at 85° C. for 45 minutes. The reaction mixture was allowed to cool to ambient temperature, water (15 mL) was added and the precipitate was collected by filtration, washed with water (10 mL) and air dried to afford the desired product as a yellow solid, which was used without further purification. m/z (ESI+) (M−H)⁻=517.46.

### Example 3

### (1r,4r)-4-(3-Fluoro-4-(5-(4-(trifluoromethoxyl)phenylamino)-1,3,4-oxadiazole-2-carboxamido)phenyl)cyclohexanecarboxylic acid

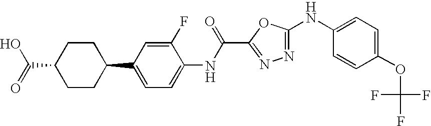

Sodium hydroxide (2M; 3.56 mL, 7.12 mmol) was added to Intermediate 3-1 (763 mg, 1.42 mmol) in MeOH (25 mL). The resulting solution was stirred for 16 hours. The reaction mixture was evaporated and the aqueous residue (20 mL) was adjusted to pH 2 with 2M HCl (5 mL). The suspension was filtered and dried to afford the crude product.

The crude product was purified by crystallisation from AcOH to afford the title compound (393 mg, 54.3%) as a white crystalline solid.

¹H NMR (400.13 MHz, DMSO-d₆) δ 1.38-1.55 (4H, m), 1.78-1.90 (2H, m), 1.90-2.05 (2H, m), 2.20-2.30 (1H, m), 2.40-2.60 (1H, m), 7.10 (1H, dd), 7.18 (1H, dd), 7.40 (1H, s), 7.42 (1H, s), 7.45 (1H, t), 7.69 (2H, dd), 10.66 (1H, s), 11.19 (1H, s), 12.02 (1H, s) m/z (ES+) (M+H)+=509.43.

### Intermediate 3-1: (1r,4r)-Ethyl 4-(3-fluoro-4-(5-(4-(trifluoromethoxy)-phenylamino)-1,3,4-oxadiazole-2-carboxamido)phenyl)cyclohexanecarboxylate

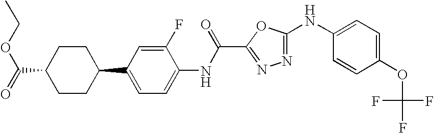

To a solution of Intermediate 1-2 (500 mg, 1.42 mmol) in DMF (10 mL) was added 1-isothiocyanato-4-(trifluoromethoxy)benzene (0.277 mL, 1.71 mmol). The resulting mixture was stirred at 45° C. for 45 minutes, 1-(3-dimethylaminopropyl)-3-ethylcarbodiimide hydrochloride (396 mg, 2.06 mmol) was added and the mixture was stirred at 85° C. for 45 minutes. The reaction mixture was allowed to cool to ambient temperature, water (15 mL) was added and the precipitate was collected by filtration, washed with water (10 mL) and air dried to afford the desired product as a yellow solid, which was used without further purification. m/z (ESI+) (M+H)+=537.47.

### Example 4

### (1r,4r)-4-(4-(5-(3-Chlorophenylamino)-1,3,4-oxadiazole-2-carboxamido)-3-fluorophenyl)cyclohexanecarboxylic acid

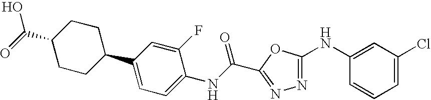

Sodium hydroxide (2M; 3.56 mL, 7.12 mmol) was added to Intermediate 4-1 (693 mg, 1.42 mmol) in MeoH (25 mL). The resulting solution was stirred for 16 hours.
The reaction mixture was evaporated and the aqueous residue (20 mL) was adjusted to pH 2 with 2M HCl (5 mL). The suspension was filtered and dried to afford the crude product. The crude product was purified by crystallisation from AcOH to afford the title compound (303 mg, 46.4%) as a white crystalline solid.

¹H NMR (400.13 MHz, DMSO-d₆) δ 1.38-1.55 (4H, m), 1.78-1.90 (2H, m), 1.90-2.05 (2H, m), 2.20-2.30 (1H, m), 2.40-2.60 (1H, m), 7.11 (2H, ddd), 7.18 (1H, dd), 7.41 (1H, t), 7.46 (1H, d), 7.50 (1H, dq), 7.73 (1H, t), 10.67 (1H, s), 11.22 (1H, s), 12.02 (1H, s). m/z (ES+) (M+H)+=459.46.

### Intermediate 4-1: (1r,4r)-Ethyl 4-(4-(5-(3-chlorophenylamino)-1,3,4-oxadiazole-2-carboxamidol-3-fluorophenyl)cyclohexanecarboxylate

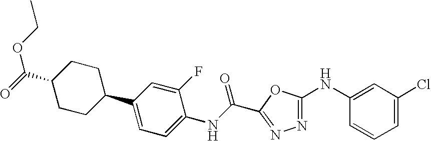

To a solution of Intermediate 1-2 (500 mg, 1.42 mmol) in DMF (10 mL) was added 1-chloro-3-isothiocyanatobenzene (0.224 mL, 1.71 mmol). The resulting mixture was stirred at 45° C. for 45 minutes, 1-(3-dimethylaminopropyl)-3-ethylcarbodiimide hydrochloride (396 mg, 2.06 mmol) was added and the mixture was stirred at 85° C. for 45 minutes. The reaction mixture was allowed to cool to ambient temperature, water (15 mL) was added and the precipitate was collected by filtration, washed with water (10 mL) and air dried to afford the desired product as a yellow solid, which was used without further purification. m/z (ESI+) (M−H)−=485.43.

### Example 5

### (1r,4r)-4-(4-(5-(3,4-Difluorophenylamino)-1,3,4-oxadiazole-2-carboxamido)-3-fluorophenyl)cyclohexanecarboxylic acid

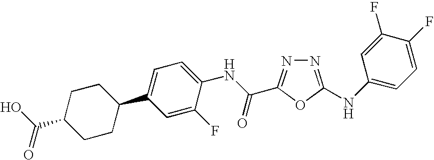

To a suspension of Intermediate 5-1 (3.63 g, 7.43 mmol) in MeOH (100 mL) and THF (50.0 mL) was added 2N sodium hydroxide (18.58 mL, 37.16 mmol). The resulting solution was stirred at ambient temperature for 3 days. The reaction mixture was adjusted to pH 4 with 1N citric acid and the suspension was evaporated to remove the organic solvents. The precipitate was collected by filtration, washed with water (50 mL) and air dried to afford the desired product as a white solid, this was slurried in water (90 mL) at ambient temperature for 3 hours. The suspension was collected by filtration, washed with water (20 mL) and air dried and then dried in the vacuum oven at 50° C. over 2 days to afford the title compound (3.30 g, 96%) as a white solid.

¹H NMR (400 MHz, DMSO) δ 1.40-1.54 (4H, m), 1.77-1.90 (2H, m), 1.92-2.08 (2H, m), 2.23-2.30 (1H, m), 2.51-2.59 (1H, m), 7.09-7.11 (1H, m), 7.17-7.20 (1H, m), 7.33-7.36 (1H, m), 7.43-7.51 (2H, m), 7.66-7.71 (1H, m), 10.67 (1H, s), 11.23 (1H, s), 12.02 (1H, s); m/z (ES−) (M−H)⁻ 459.

The X-ray powder diffraction spectra for Example 5 showed the material to be crystalline with a melting point of 286.2° C. (onset). This material is termed Form A. Further measurements showed the material to be crystalline with a melting point of 270-300° C. (onset). DSC analysis of Form A showed initial events before melting due to weight loss.

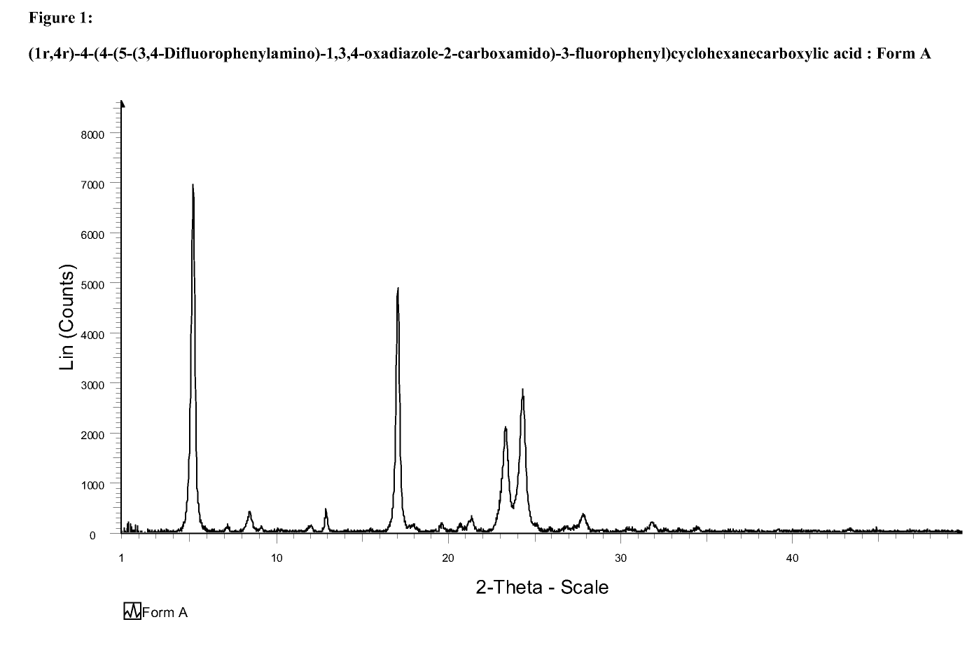

The material termed Form A is, according to the present investigation, characterized in providing an X-ray powder diffraction pattern (see FIG. 1) exhibiting substantially the following d-values and intensities for the ten most prominent peaks shown in Table A. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE A
Ten most prominent X-Ray Powder Diffraction peaks for Form A

| d-value | | Relative |
|---|---|---|
| (Angstrom) | Intensity % | Intensity |
| 17.3 | 100.0 | vs |
| 5.2 | 70.3 | vs |
| 3.66 | 39.4 | vs |
| 3.81 | 30.3 | vs |
| 6.9 | 6.7 | m |
| 10.5 | 5.9 | m |
| 3.21 | 4.9 | m |
| 4.19 | 3.2 | m |
| 2.81 | 2.9 | m |
| 4.53 | 2.4 | w |

vs = very strong;
s = strong;
m = medium;
w = weak

The peaks, identified with d-values calculated from the Bragg formula and intensities, have been extracted from the diffractogram of Form A. The relative intensities have been estimated without any divergence slit conversion and are derived from diffractograms measured with variable slits.

### Example 5B

By slurrying Example 5 material in methanol, a further form (Form B) was produced. Approximately 20 mg of material was placed in a vial with a magnetic flea, and approximately 2 ml of methanol added, the vial was then sealed tightly with a cap and left to stir on a magnetic stirrer plate. After 3 days, the sample was removed from the plate, the cap taken off and the slurry left to dry under ambient conditions before it was analysed by XRPD and DSC. This form (Form B) was determined to be crystalline by XRPD, with a melting point of 288.6° C. (onset).

Using CuKa radiation: 6.2 and 27.6 an X-ray powder diffraction pattern for Form B was obtained with the ten most prominent peaks shown in Table B-1:

TABLE B-1
Ten most prominent X-Ray Powder Diffraction peaks for Form B

| Angle 2- | | Relative |
|---|---|---|
| Theta (2θ) | Intensity % | Intensity |
| 16.150 | 100.0 | vs |
| 27.561 | 57.6 | vs |
| 25.526 | 49.3 | vs |
| 20.218 | 34.0 | vs |
| 6.612 | 24.8 | s |
| 26.722 | 20.6 | s |
| 9.808 | 17.0 | s |
| 27.019 | 17.0 | s |
| 31.520 | 15.5 | s |
| 23.881 | 14.0 | s |

vs = very strong;
s = strong

According to the present invention there is provided a crystalline form, Form B, which has an X-ray powder diffraction pattern with at least two specific peaks at about 2-theta=16.2° and 27.6°, wherein said values may be plus or minus 0.5° 2-theta.

According to the present invention there is provided a crystalline form, Form B, which has an X-ray powder diffraction pattern with specific peaks at 2-theta=16.2, 27.6, 25.5, 20.2, 6.6, 26.7, 9.8, 27.0, 31.5, 23.9° wherein said values may be plus or minus 0.5° 2-theta.

DSC analysis of Form B showed an initial event with an onset at 209.9° C. and a peak at 219.0° C. followed by a subsequent melt with an onset of 288.6° C. and a peak at 293.4° C. Further DSC analysis of Form B showed initial events before melting due to weight loss followed by a subsequent melt with an onset of 280-310° C. (onset).

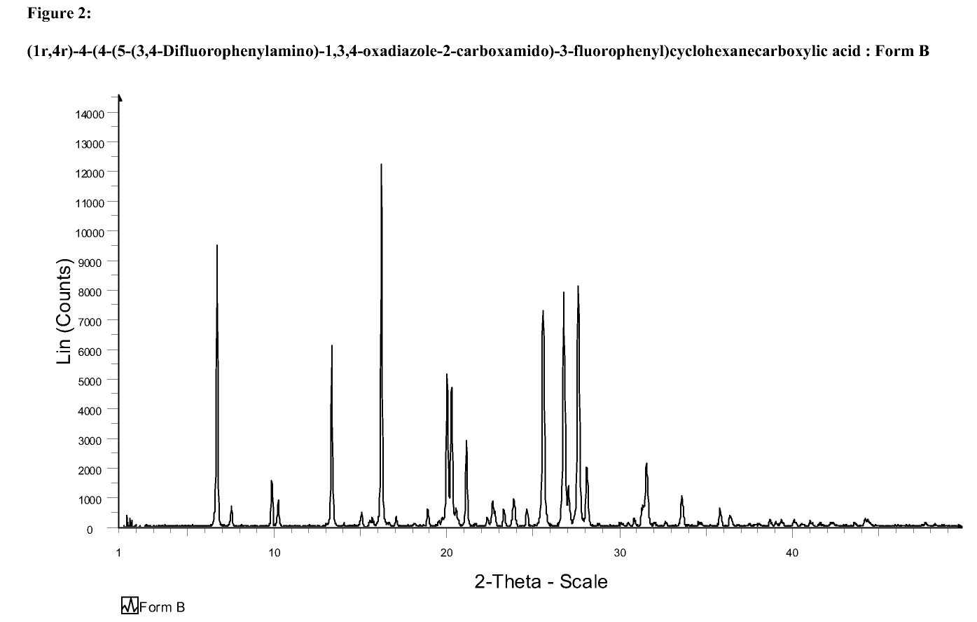

This material (Form B) is, according to the present investigation, characterized in providing an X-ray powder diffraction pattern (see FIG. 2) exhibiting substantially the following d-values and intensities for the ten most prominent peaks shown in Table B-2. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE B-2
Ten most prominent X-Ray Powder Diffraction peaks for Form B

| d-value | | Relative |
|---|---|---|
| (Angstrom) | Intensity % | Intensity |
| 5.5 | 100.0 | vs |
| 13.3 | 77.7 | vs |
| 3.50 | 59.7 | vs |
| 3.33 | 56.7 | vs |
| 6.6 | 49.9 | vs |
| 3.22 | 40.3 | vs |
| 4.19 | 19.7 | s |
| 2.84 | 16.0 | s |
| 3.30 | 11.2 | s |
| 4.41 | 10.0 | s |

vs = very strong;
s = strong

The peaks, identified with d-values calculated from the Bragg formula and intensities, have been extracted from the diffractogram of Form B. The relative intensities have been estimated without any divergence slit conversion and are derived from diffractograms measured with variable slits.

### Alternative preparation of Example 5

Sodium hydroxide (587 mL) was added drop wise over 30 mins to Intermediate 5-1 (114.7 g) in ethanol (2300 mL) at 20° C. The resulting solution was stirred at 20° C. for 24 hours. 1M citric acid was added until pH 4 and the slurry filtered, washed with water (3000 mL) and dried in the vac. oven at 50° C. over P₂O₅ until constant weight. The crude product was purified by crystallisation from EtOH, the solid was filtered and dried in the vacuum oven at 45° C. for 48 hours to provide 55.5 g the desired product as a mixture of polymorphic crystal forms as shown by XRPD. This material was slurried in methanol (550 mL) over the weekend in the presence of a seed crystal of Form B (see above) to convert it all into this form. The cream slurry was filtered and the solid dried in the vac oven for 48 h at 40° C. to provide (1r,4r)-4-(4-(5-(3,4-difluorophenylamino)-1,3,4-oxadiazole-2-carboxamido)-3-fluorophenyl)cyclohexanecarboxylic acid (54.5 g) as a cream solid.

¹H NMR (400 MHz, DMSO) δ 1.37-1.58 (4H, m), 1.75-1.91 (2H, m), 1.93-2.09 (2H, m), 2.20-2.36 (1H, m), 2.51-2.63 (1H, m), 7.06-7.15 (1H, m), 7.16-7.25 (1H, m), 7.30-7.39 (1H, m), 7.39-7.55 (2H, m), 7.65-7.76 (1H, m), 10.75 (1H, s), 11.29 (1H, s), 12.10 (1H, s).

N.B. The compound (1r,4r)-4-(4-(5-(3,4-Difluorophenylamino)-1,3,4-oxadiazole-2-carboxamido)-3-fluorophenyl)cyclohexanecarboxylic acid (see Example 5) may be alternatively named trans-4-(4-(5-(3,4-difluorophenylamino)-1,3,4-oxadiazole-2-carboxamido)-3-fluorophenyl)cyclohexanecarboxylic acid or trans-4-{4-[({5-[(3,4-difluorophenyl)amino]-1,3,4-oxadiazol-2-yl}carbonyl)amino]-3-fluorophenyl}cyclohexanecarboxylic acid.

### Intermediate 5-1: (1r,4r)-Ethyl 4-(4-(5-(3,4-difluorophenylamino)-1,3,4-oxadiazole-2-carboxamido)-3-fluorophenyl)cyclohexanecarboxylate

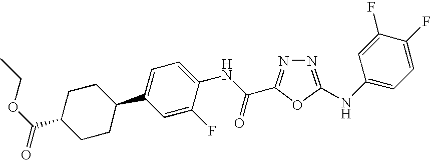

To a solution of Intermediate 1-2 (3.01 g, 8.57 mmol) in DMF (134 mL) was added 3,4-difluorophenyl isothiocyanate (1.760 g, 10.28 mmol). The resulting mixture was stirred at 45° C. for 45 minutes, 1-(3-dimethylaminopropyl)-3-ethylcarbodiimide hydrochloride (2.381 g, 12.42 mmol) was added and the mixture was stirred at 85° C. for 45 minutes. The reaction mixture was allowed to cool to ambient temperature, water (140 mL) was added and the precipitate was collected by filtration, washed with water (10 mL) and air dried to afford the title compound (3.63 g, 87%) as a cream solid, which was used without further purification.

¹H NMR (400 MHz, DMSO) δ 1.19 (3H, t), 1.42-1.55 (4H, m), 1.81-1.86 (2H, m), 1.98-2.00 (2H, m), 2.31-2.39 (1H, m), 2.51-2.55 (1H, m), 4.07 (2H, q), 7.08-7.11 (1H, m), 7.16-7.20 (1H, m), 7.33-7.36 (1H, m), 7.43-7.51 (2H, m), 7.66-7.72 (1H, m), 10.67 (1H, s), 11.23 (1H, s); m/z (M+H)⁺ 489.

### Alternative Preparation of Intermediate 5-1

1,2-difluoro-4-isothiocyanatobenzene (50.1 g) was added drop wise to Intermediate 1-2 (85.7 g) in DMF (1275 mL) at 20° C. The resulting solution was stirred at 45° C. for 30 minutes. 1-(3-Dimethylaminopropyl)-3-ethylcarbodiimide hydrochloride (67.8 g) was charged and the reaction heated to 85° C. After 30 mins the reaction was cooled to ambient temperature. Water (1720 mL) was added drop-wise and the precipitate was collected by filtration, washed with water (3000 mL) and dried in the vac oven at 50° C. over P₂O₅ until constant weight to afford (1r,4r)-ethyl 4-(4-(5-(3,4-difluorophenylamino)-1,3,4-oxadiazole-2-carboxamido)-3-fluorophenyl)cyclo-hexanecarboxylate (111 g, 93%) as a yellow solid, which was used without further purification.

¹H NMR (400 MHz, DMSO) δ 1.20 (3H, t), 1.40-1.59 (4H, m), 1.78-1.91 (2H, m), 1.93-2.06 (2H, m), 2.30-2.41 (1H, m), 2.51-2.62 (1H, m), 4.07 (2H, q), 7.08-7.13 (1H, m), 7.20 (1H, dd), 7.32-7.38 (1H, m), 7.41-7.56 (2H, m), 7.70 (1H, ddd), 10.76 (1H, s), 11.29 (1H, s).

### Alternative Preparation of Example 5: Example 5C

To a suspension of Intermediate 5-1 (6.98 g, 14.29 mmol) in MeOH (700 ml) was added 2N sodium hydroxide (35.7 ml, 71.45 mmol) and the resulting solution was stirred at ambient temperature for 24 hours. The reaction was incomplete so further 2N sodium hydroxide (20 ml) and THF (100 ml) were added and the mixture was stirred at ambient temperature for a further 2 days. The reaction mixture was adjusted to pH 5 with 1N citric acid and the suspension was evaporated to remove the organic solvents. The suspension was adjusted to pH 4 with 1N citric acid and the precipitate was collected by filtration, washed with water (150 ml) and air dried to afford crude product. The crude solid was suspended in ACN (60 ml) and heated to 100° C. for 10 minutes and the suspension was collected by filtration and dried under vacuum to give impure product (5.38 g) as a white solid. This process was repeated with by ACN (70 ml) and the suspension was collected by filtration and dried under vacuum to give (1r,4r)-4-(4-(5-(3,4-difluorophenylamino)-1,3,4-oxadiazole-2-carboxamido)-3-fluorophenyl)cyclohexanecarboxylic acid (5.08 g, 77%) as a white solid.

¹H NMR (400.13 MHz, DMSO-d₆) δ 1.40-1.53 (4H, m), 1.77-1.90 (2H, m), 1.92-2.08 (2H, m), 2.23-2.30 (1H, m), 2.51-2.59 (1H, m), 7.10 (1H, d), 7.18 (1H, d), 7.33 (1H, d), 7.43-7.50 (2H, m), 7.66-7.71 (1H, m), 10.67 (1H, s), 11.23 (1H, s) m/z (ES−) (M−H)⁻=459.

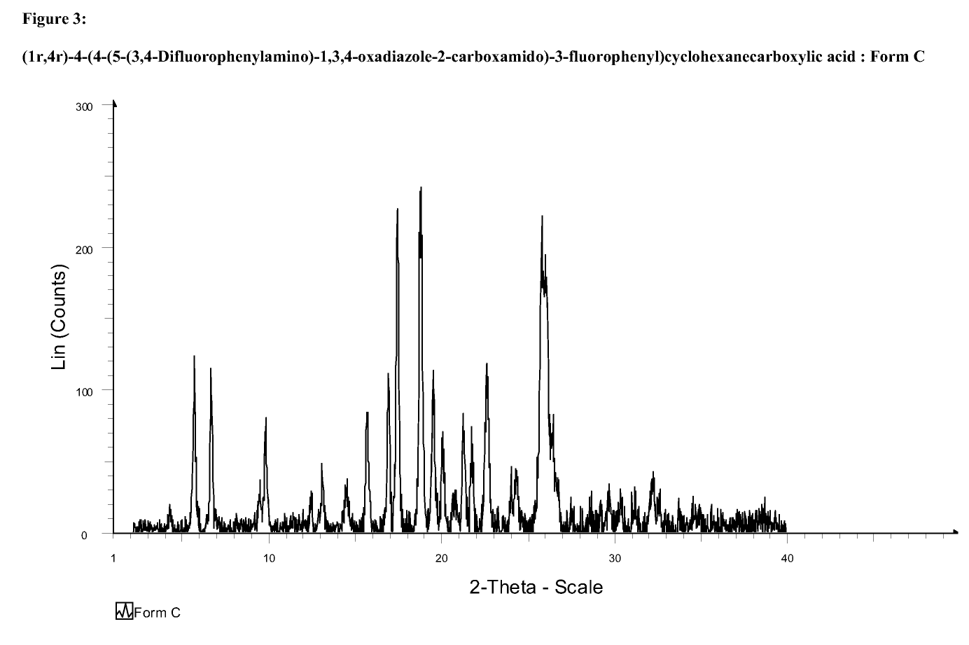

This form (Form C) was determined to be crystalline by XRPD. DSC analysis of Form C showed initial events before melting due to weight loss followed by a subsequent melt with an onset of 260-390° C. (onset). This material (Form C) is, according to the present investigation, characterized in providing an X-ray powder diffraction pattern (see FIG. 3) exhibiting substantially the following d-values and intensities for the twelve most prominent peaks shown in Table C. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE C
Twelve most prominent X-Ray Powder Diffraction peaks for Form C

| d-value | | Relative |
|---|---|---|
| (Angstrom) | Intensity % | Intensity |
| 4.72 | 100 | vs |
| 5.1 | 93.7 | vs |
| 3.45 | 91.5 | vs |
| 3.43 | 80.5 | vs |
| 15.6 | 51.1 | vs |
| 3.93 | 48.8 | vs |
| 13.3 | 47.3 | vs |
| 4.55 | 46.8 | vs |
| 5.2 | 45.9 | vs |
| 5.7 | 34.6 | vs |
| 9.0 | 33.0 | vs |
| 4.1 | 30.5 | vs |

vs = very strong

The peaks, identified with d-values calculated from the Bragg formula and intensities, have been extracted from the diffractogram of Form C. The relative intensities have been estimated without any divergence slit conversion and are derived from diffractograms measured with variable slits.

### Alternative Preparation of Example 5: Example 5D

See Scheme D below

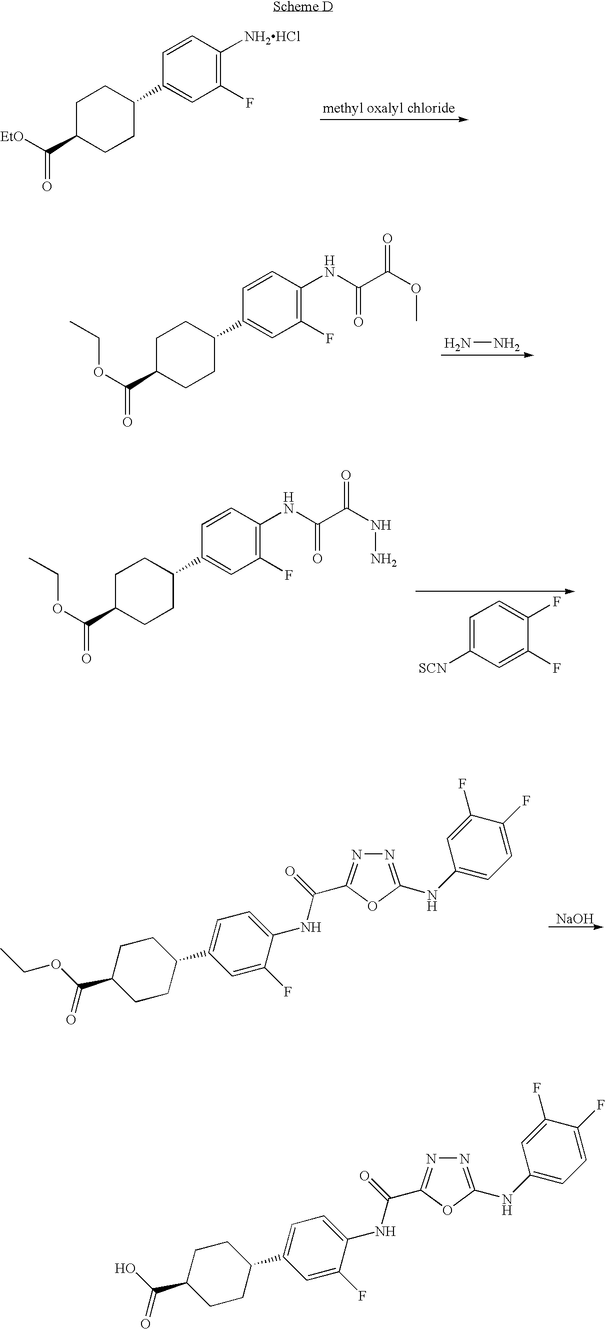

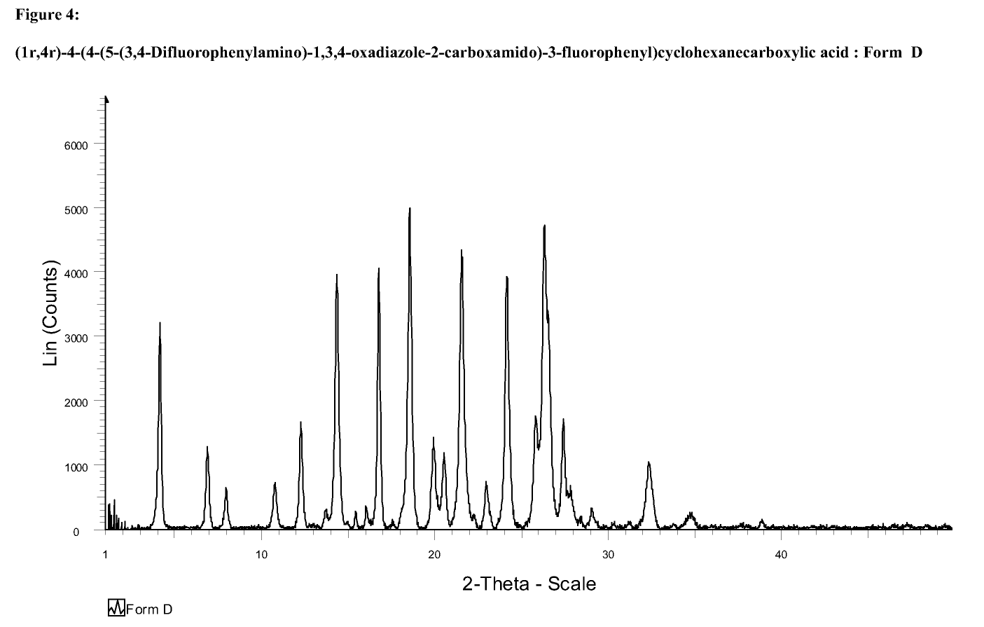

To a suspension of Intermediate 5-1 (10 g, 20.47 mmol) in EtOH (150 ml) was added a solution of sodium hydroxide (4.34 g) in water (150 ml) over a period of 15 minutes at 25° C. The resulting mixture was stirred at the same temperature for 2 h. After completion of the reaction, the mixture was acidified with a solution of citric acid (10.43 g) in water (100 ml) and stirred for 1 hour. The creamy white solid was filtered and slurried in water (100 ml) at 25° C. for 1 h. The solid was filtered and dried in vacuum oven at 50° C. for 12 h to give (1r,4r)-4-(4-(5-(3,4-difluorophenylamino)-1,3,4-oxadiazole-2-carboxamido)-3-fluorophenyl)cyclohexanecarboxylic acid (8.6 g, 91%) as white solid. The XRPD of the solid showed Form D. The material was further purified by slurring with isopropanol (100 ml) at 75° C. for 5 h. The resultant suspension was cooled to 25° C. and filtered. The material was dried in vacuum oven at 50° C. for 12 h to afford 7.7 g of creamy white solid whose XRPD was consistent with Form D. DSC analysis of Form D showed no initial events before melting due to weight loss and showed a melt with an onset of 260-310° C. (onset). This material (Form D) is, according to the present investigation, characterized in providing an X-ray powder diffraction pattern (see FIG. 4) exhibiting substantially the following d-values and intensities for the twelve most prominent peaks shown in Table D. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE D
Twelve most prominent X-Ray Powder Diffraction peaks for Form D

| d-value | | Relative |
|---|---|---|
| (Angstrom) | Intensity % | Intensity |
| 4.78 | 100 | vs |
| 3.38 | 90.5 | vs |
| 4.12 | 86.9 | vs |
| 5.29 | 81.2 | vs |
| 6.2 | 79.1 | vs |
| 3.7 | 78.4 | vs |
| 21.6 | 64 | vs |
| 3.44 | 35 | vs |
| 3.25 | 34 | vs |
| 7.2 | 33.1 | vs |
| 12.9 | 25.4 | vs |
| 2.76 | 20.6 | s |

vs = very strong;
s = strong

The peaks, identified with d-values calculated from the Bragg formula and intensities, have been extracted from the diffractogram of Form D. The relative intensities have been estimated without any divergence slit conversion and are derived from diffractograms measured with variable slits.

### Alternative Preparation of Example 5: Example 5E

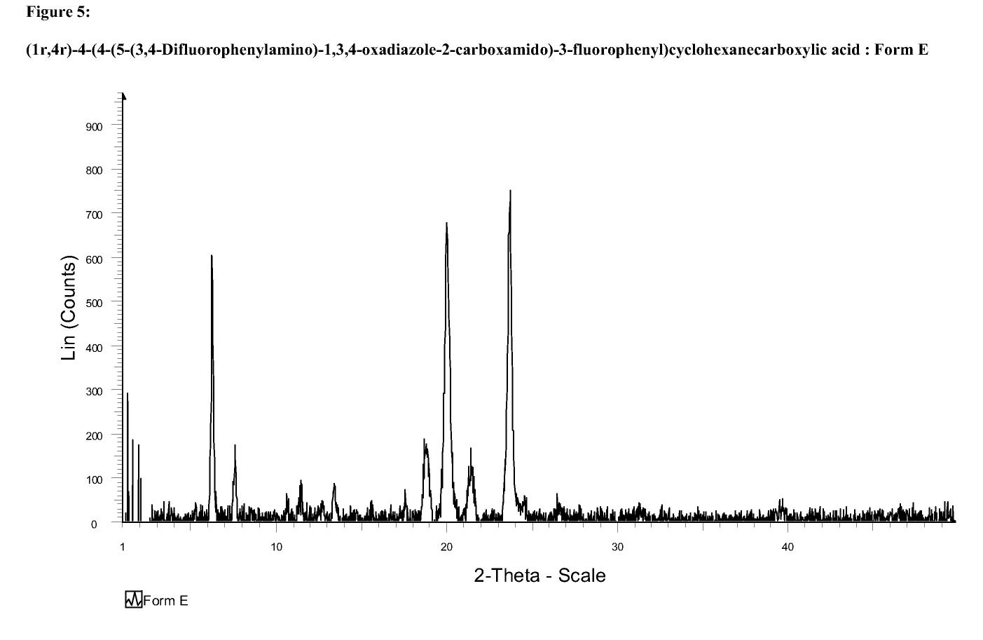

An approximate amount of (1r,4r)-4-(4-(5-(3,4-difluorophenylamino)-1,3,4-oxadiazole-2-carboxamido)-3-fluorophenyl)cyclohexanecarboxylic acid (approximate 10 mg) of the known Form B was heated to 242° C. for 1 h in a temperature chamber fitted in the powder x-ray diffractometer. The PXRD of the resulting solid showed Form E, which had a melting onset of 260-300° C. Repeated experiments showed that the same form was obtained when heating the solid to a temperature above 240° C. This material (Form E) is, according to the present investigation, characterized in providing an X-ray powder diffraction pattern (see FIG. 5) exhibiting substantially the following d-values and intensities for the ten most prominent peaks shown in Table E. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE E
Ten most prominent X-Ray Powder Diffraction peaks for Form E

| d-value | | Relative |
|---|---|---|
| (Angstrom) | Intensity % | Intensity |
| 3.75 | 100 | vs |
| 4.43 | 90.1 | vs |
| 14.2 | 80.6 | vs |
| 4.72 | 23.4 | s |
| 11.7 | 23.1 | s |
| 4.15 | 22.1 | s |
| 7.7 | 12.3 | s |
| 6.6 | 11.3 | s |
| 5.1 | 9.3 | m |
| 3.35 | 5.0 | m |

vs = very strong;
s = strong;
m = medium

The peaks, identified with d-values calculated from the Bragg formula and intensities, have been extracted from the diffractogram of Form E. The relative intensities have been estimated without any divergence slit conversion and are derived from diffractograms measured with variable slits.

### Alternative Preparation of Example 5: Example 5F

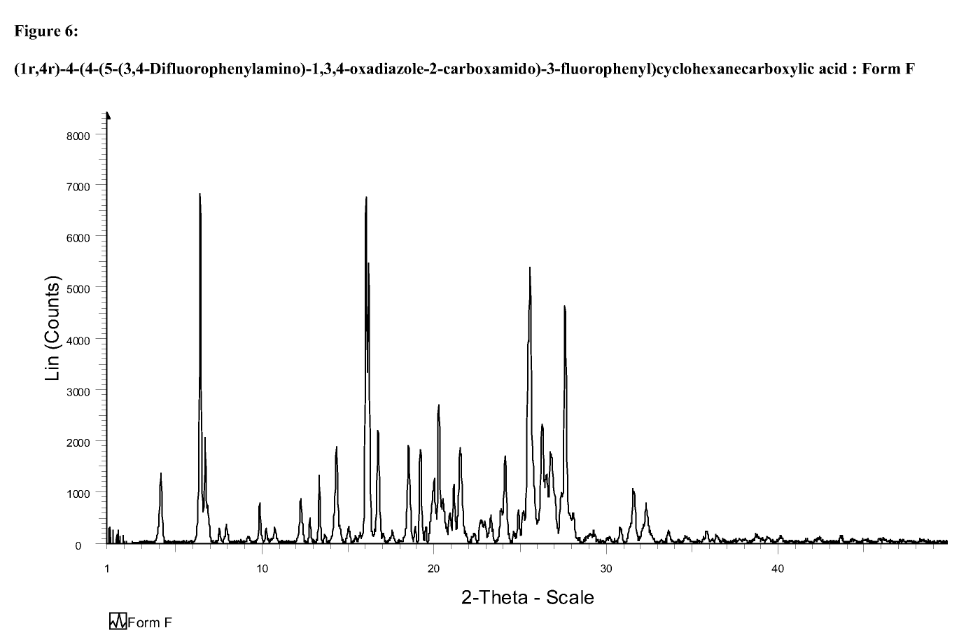

Sodium hydroxide (5850 ml, 11700.06 mmol) was added dropwise over 30 mins to Intermediate 5-1 (1143 g, 2340.01 mmol) in ethanol (20 vol %) (22860 ml) at 20° C. The resulting yellow/green solution was stirred at 20° C. for 24 hours. LC-MS showed no starting material, and to the yellow solution was added 1M citric acid until pH 4.7 (10000 ml) keeping the reaction temperature at 20° C. and the slurry was filtered, washed with ethanol (4500 ml, 4 vol), water (28000 ml, 25 vol). The white solid was dried in the vac. oven at 50° C. over P₂O₅ until constant weight. There was obtained (1r,4r)-4-(4-(5-(3,4-difluorophenylamino)-1,3,4-oxadiazole-2-carboxamido)-3-fluorophenyl)-cyclohexanecarboxylic acid (969 g, 90%) as a white solid.
HPLC analysis showed purity at 99.3%. The PXRD of the resulting solid showed Form F, which had a melting onset of 280-310° C. XRPD analysis showed that this material is, according to the present investigation, characterized in providing an X-ray powder diffraction pattern (see FIG. 6) exhibiting substantially the following d-values and intensities for the fourteen most prominent peaks shown in Table F. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE F
Fourteen most prominent X-Ray Powder Diffraction peaks for Form F

| d-value | | Relative |
|---|---|---|
| (Angstrom) | Intensity % | Intensity |
| 13.8 | 100 | vs |
| 5.5 | 99.0 | vs |
| 3.48 | 78.9 | vs |
| 3.22 | 67.6 | vs |
| 4.38 | 39.3 | vs |
| 3.39 | 33.8 | vs |
| 5.3 | 32.0 | vs |
| 13.2 | 30.1 | vs |
| 4.79 | 27.6 | vs |
| 6.18 | 27.5 | vs |
| 4.12 | 27.2 | vs |
| 4.62 | 26.4 | vs |
| 3.32 | 25.2 | vs |
| 3.68 | 24.7 | vs |

vs = very strong

The peaks, identified with d-values calculated from the Bragg formula and intensities, have been extracted from the diffractogram of Form F. The relative intensities have been estimated without any divergence slit conversion and are derived from diffractograms measured with variable slits.

### Example 5G

### (1r,4r)-4-(4-(5-(3,4-Difluorophenylamino)-1,3,4-oxadiazole-2-carboxamido)-3-fluorophenyl)cyclohexanecarboxylic acid nanosuspensions

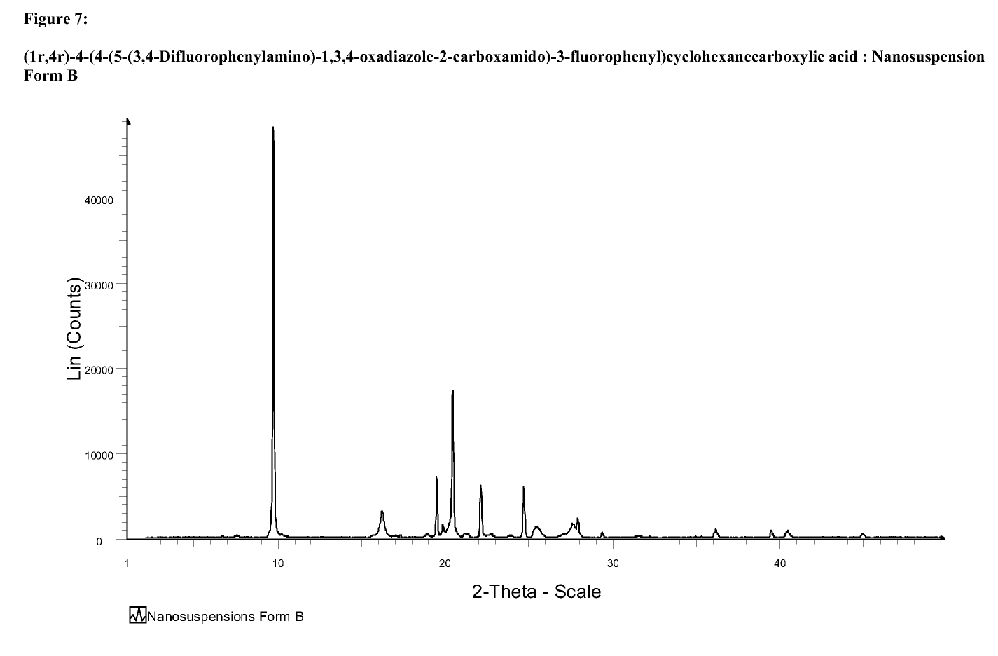

The vehicle used for preparation of nanosuspension of Form B was Polyvinylpyrrolidone(0.67% w/v)/Aerosol OT(0.033% w/v)/Mannitol (5% w/v) in de-ionised water. The vehicle used is typically prepared in the following way (e.g 100 ml): Polyvinylpyrrolidone (0.67 g, Kollidone 25 grade ex. BASF), Aerosol OT-100 (0.033 g, sodium dioctyl sulphosuccinate ex. Cyclex Industries) and Mannitol (5 g) was weighed into a volumetric flask. De-ionised water approximate 70 ml) was added and sonicated until a solution is formed. The volume was made up with de-ionised water to produce a solution containing Polyvinylpyrrolidone (0.67% w/v), Aerosol OT (0.033% w/v) and Mannitol (5% w/v) in de-ionised water.
The required amount of compound to produce the final concentration of drug in suspension was weighed in to a suitable vessel and a small amount of vehicle was added to wet the compound. The resulting slurry was mixed using sonication.
The nanosuspension of Form B was milled according to the method described herein (Nanosuspension Preparation). XRPD analysis of the resulting nanosuspension is, according to the present investigation, characterized in providing an X-ray powder diffraction pattern (see FIG. 7) exhibiting substantially the following d-values and intensities for the ten most prominent peaks shown in Table G. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE G
Twelve most prominent X-Ray Powder Diffraction peaks
for nanosuspension of Form B

| d-value | | Relative |
|---|---|---|
| (Angstrom) | Intensity % | Intensity |
| 9.1 | 100 | vs |
| 4.34 | 35.9 | vs |
| 4.55 | 15.1 | s |
| 4.01 | 12.8 | s |
| 3.60 | 12.5 | s |
| 5.5 | 6.5 | m |
| 3.19 | 4.9 | m |
| 3.23 | 3.5 | m |
| 4.47 | 3.3 | m |
| 3.5 | 2.8 | w |
| 2.48 | 2.0 | w |

vs = very strong;
s = strong;
m = medium;
w = weak

The peaks, identified with d-values calculated from the Bragg formula and intensities, have been extracted from the diffractogram of this nanosuspension. The relative intensities have been estimated without any divergence slit conversion and are derived from diffractograms measured with variable slits.

### Alternative Preparation: Example 5H

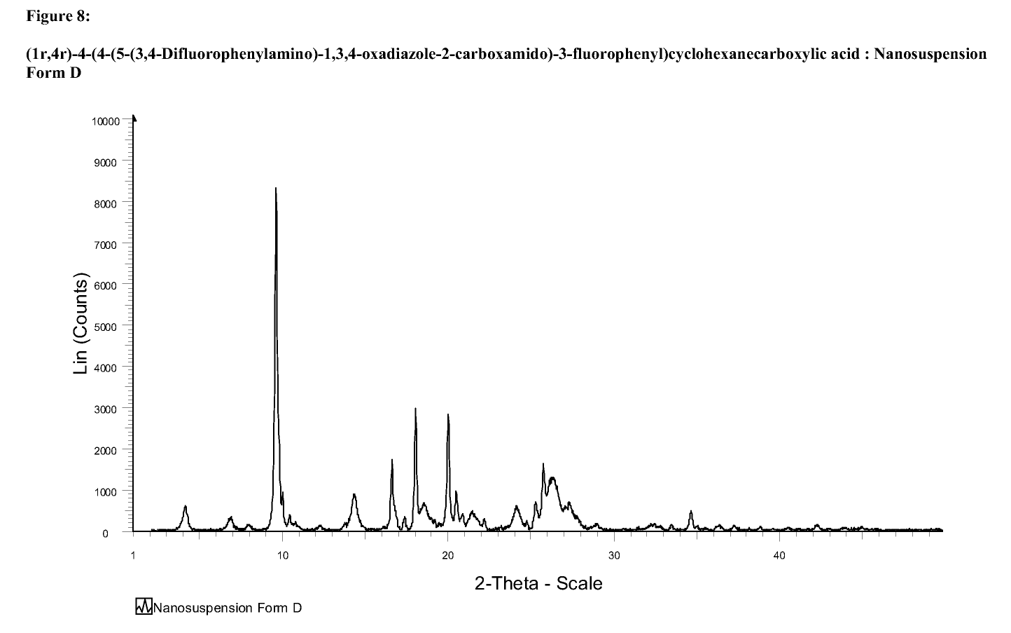

The vehicle used for preparation of nanosuspension of Form D was Polyvinylpyrrolidone(0.67% w/v)/Aerosol OT(0.033% w/v)/Mannitol (5% w/v) in de-ionised water. See Example 5G for vehicle preparation instructions.
The required amount of compound to produce the final concentration of drug in suspension was weighed in to a suitable vessel and a small amount of vehicle was added to wet the compound. The resulting slurry was mixed using sonication.
The nanosuspension of Form D was milled according to the method described herein (Nanosuspension Preparation). XRPD analysis of the resulting nanosuspension is, according to the present investigation, characterized in providing an X-ray powder diffraction pattern (see FIG. 8) exhibiting substantially the following d-values and intensities for the ten most prominent peaks shown in Table H. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE H
Ten most prominent X-Ray Powder Diffraction peaks
for nanosuspension of Form D

| d-value | | Relative |
|---|---|---|
| (Angstrom) | Intensity % | Intensity |
| 9.2 | 100 | vs |
| 4.92 | 35.5 | vs |
| 4.44 | 33.9 | vs |
| 5.3 | 20.6 | s |
| 3.45 | 19.6 | s |
| 3.38 | 15.1 | s |
| 4.33 | 11.5 | s |
| 8.8 | 11.2 | s |
| 6.2 | 10.6 | s |
| 3.51 | 8.4 | m |

vs = very strong;
s = strong;
m = medium

The peaks, identified with d-values calculated from the Bragg formula and intensities, have been extracted from the diffractogram of this nanosuspension. The relative intensities have been estimated without any divergence slit conversion and are derived from diffractograms measured with variable slits.

### Alternative Preparation: Example 5I

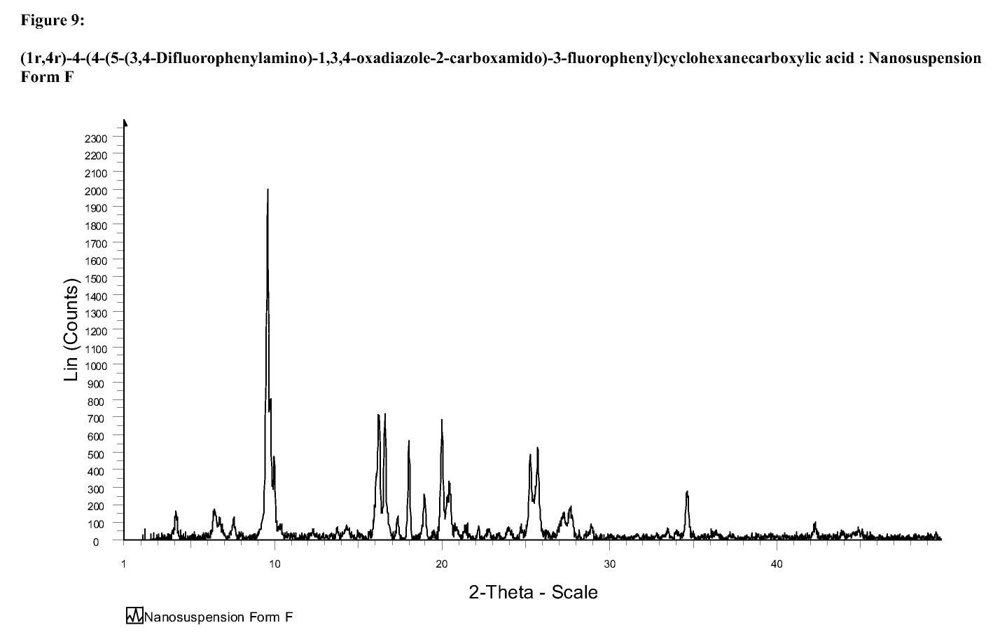

The vehicle used for preparation of nanosuspension of Form F was Polyvinylpyrrolidone(0.67% w/v)/Aerosol OT(0.033% w/v)/Mannitol (5% w/v) in de-ionised water. See Example 5G for vehicle preparation instructions. The required amount of compound to produce the final concentration of drug in suspension was weighed in to a suitable vessel and a small amount of vehicle was added to wet the compound. The resulting slurry was mixed using sonication.
The nanosuspension of form F was milled similarly according to the method described herein (Nanosuspension Preparation). XRPD analysis of the resulting nanosuspension is, according to the present investigation, characterized in providing an X-ray powder diffraction pattern (see FIG. 9) exhibiting substantially the following d-values and intensities for the ten most prominent peaks shown in Table I. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE I
Ten most prominent X-Ray Powder Diffraction peaks
for nanosuspension of Form F

| d-value | | Relative |
|---|---|---|
| (Angstrom) | Intensity % | Intensity |
| 9.3 | 100 | vs |
| 5.3 | 35.7 | vs |
| 5.5 | 35.6 | vs |
| 4.44 | 34.0 | vs |
| 4.93 | 28.1 | vs |
| 3.46 | 26.0 | vs |
| 3.52 | 24.0 | s |
| 8.9 | 23.5 | s |
| 4.34 | 16.3 | s |
| 2.59 | 13.6 | s |
| 4.69 | 12.6 | s |

vs = very strong;
s = strong

The peaks, identified with d-values calculated from the Bragg formula and intensities, have been extracted from the diffractogram of this nanosuspension. The relative intensities have been estimated without any divergence slit conversion and are derived from diffractograms measured with variable slits.

### Example 5J

### Milling of (1r,4r)-4-(4-(5-(3,4-Difluorophenylamino)-1,3,4-oxadiazole-2-carboxamido)-3-fluorophenyl)cyclohexanecarboxylic acid Form B in water

The vehicle used for preparation of nanosuspension of Form B was pure de-ionised water. The required amount of compound to produce the final concentration of drug in suspension was weighed in to a suitable vessel and a small amount of vehicle was added to wet the compound. The resulting slurry was mixed using sonication.
The nanosuspension of Form B was milled according to the method described herein (Nanosuspension Preparation). XRPD analysis of the resulting suspension is, according to the present investigation, characterized in providing an X-ray powder diffraction pattern exhibiting substantially the following d-values and intensities for the ten most prominent peaks shown in Table J. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE J
Ten most prominent X-Ray Powder Diffraction peaks
for suspension of Form B

| d-value | | Relative |
|---|---|---|
| (Angstrom) | Intensity % | Intensity |
| 5.5 | 100 | vs |
| 3.23 | 23.0 | s |
| 4.38 | 20.7 | s |
| 3.48 | 17.0 | s |
| 9.0 | 10.4 | s |
| 11.8 | 9.8 | m |
| 3.91 | 9.3 | m |
| 3.72 | 8 | m |
| 3.29 | 7.9 | m |
| 5.66 | 7.1 | m |

vs = very strong;
s = strong;
m = medium

The peaks, identified with d-values calculated from the Bragg formula and intensities, have been extracted from the diffractogram of this suspension. The relative intensities have been estimated without any divergence slit conversion and are derived from diffractograms measured with variable slits.

### Example 5K

### Milling of (1r,4r)-4-(4-(5-(3,4-Difluorophenylamino)-1,3,4-oxadiazole-2-carboxamido)-3-fluorophenyl)cyclohexanecarboxylic acid Form D in water

The vehicle used for preparation of suspension of Form D was pure de-ionised water. The required amount of compound to produce the final concentration of drug in suspension was weighed in to a suitable vessel and a small amount of vehicle was added to wet the compound. The resulting slurry was mixed using sonication. The suspension of Form D was milled according to the method described herein (Nanosuspension Preparation). XRPD analysis of the resulting suspension is, according to the present investigation, characterized in providing an X-ray powder diffraction pattern exhibiting substantially the following d-values and intensities for the ten most prominent peaks shown in Table K. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE K
Ten most prominent X-Ray Powder Diffraction peaks
for suspension of form D

| d-value | | Relative |
|---|---|---|
| (Angstrom) | Intensity % | Intensity |
| 5.5 | 100 | vs |
| 3.22 | 19.6 | s |
| 3.48 | 14.5 | s |
| 4.37 | 14.3 | s |
| 9.0 | 8.7 | m |
| 11.8 | 7.9 | m |
| 3.72 | 6.1 | m |
| 3.91 | 6.1 | m |
| 3.29 | 5.3 | m |
| 3.81 | 5.0 | m |

vs = very strong;
s = strong;
m = medium

The peaks, identified with d-values calculated from the Bragg formula and intensities, have been extracted from the diffractogram of this suspension. The relative intensities have been estimated without any divergence slit conversion and are derived from diffractograms measured with variable slits.

### Example 5L

### Preparation of Sodium salt of (1r,4r)-4-(4-(5-(3,4-Difluorophenylamino)-1,3,4-oxadiazole-2-carboxamido)-3-fluorophenyl)cyclohexanecarboxylic acid

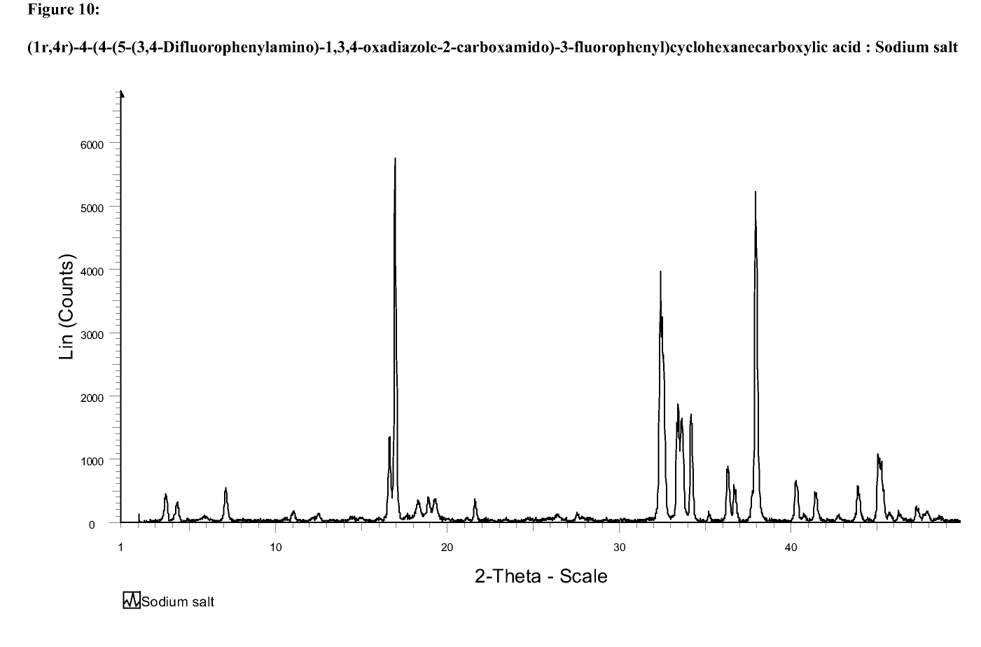

To a vial was added (1r,4r)-4-(4-(5-(3,4-Difluorophenylamino)-1,3,4-oxadiazole-2-carboxamido)-3-fluorophenyl)cyclohexanecarboxylic acid (5.7 mg) and THF (4 ml) was added. Sodium hydroxide was added in 1:1 salt:substance ratio. The resulting solution were left on stirring until dry substance had been achieved. Analysis with Raman microscopy, thermal analysis and powder X-ray diffraction indicated on salt formation. XRPD analysis of the resulting material is, according to the present investigation, characterized in providing an X-ray powder diffraction pattern (see FIG. 10) exhibiting substantially the following d-values and intensities for the fifteen most prominent peaks shown in Table L. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE L
Fifteen most prominent X-Ray Powder Diffraction peaks for
sodium salt of 1r,4r)-4-(4-(5-(3,4-Difluorophenylamino)-1,3,4-
oxadiazole-2-carboxamido)-3-fluorophenyl)cyclohexanecarboxylic acid.

| d-value | | Relative |
|---|---|---|
| (Angstrom) | Intensity % | Intensity |
| 5.2 | 100 | vs |
| 2.37 | 86.8 | vs |
| 2.76 | 66.1 | vs |
| 2.68 | 33.4 | vs |
| 2.62 | 30.3 | vs |
| 2.66 | 29.1 | vs |
| 5.3 | 23.9 | s |
| 2.01 | 17.2 | s |
| 2.47 | 15.6 | s |
| 2.23 | 11.0 | s |
| 2.06 | 10.0 | s |
| 12.49 | 9.5 | m |
| 2.44 | 8.6 | m |
| 2.18 | 8.4 | m |
| 24.6 | 7.8 | m |

vs = very strong;
s = strong;
m = medium

The peaks, identified with d-values calculated from the Bragg formula and intensities, have been extracted from the diffractogram of this salt. The relative intensities have been estimated without any divergence slit conversion and are derived from diffractograms measured with variable slits.

### Example 5M

### Preparation of Magnesium salt of (1r,4r)-4-(4-(5-(3,4-Difluorophenylamino)-1,3,4-oxadiazole-2-carboxamido)-3-fluorophenybcyclohexanecarboxylic acid

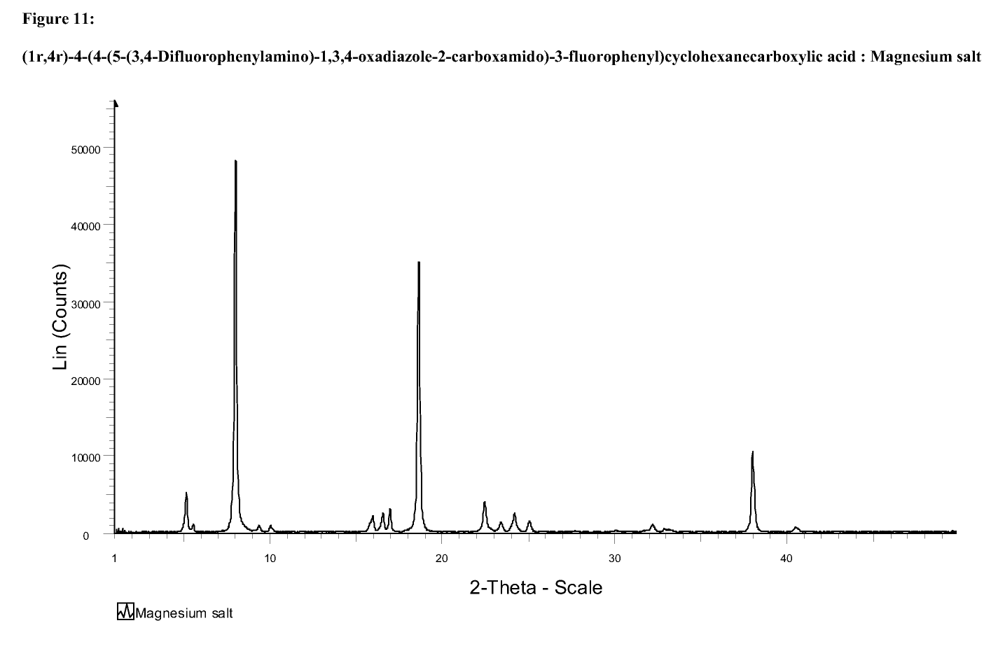

To a vial was added (1r,4r)-4-(4-(5-(3,4-Difluorophenylamino)-1,3,4-oxadiazole-2-carboxamido)-3-fluorophenyl)cyclohexanecarboxylic acid (5.7 mg) and THF (4 ml) was added. Magnesium hydroxide was added in 1:1 salt:substance ratio. The resulting solution were left on stirring until dry substance had been achieved. Analysis with Raman microscopy, thermal analysis and powder X-ray diffraction indicated on salt formation. XRPD analysis of the resulting material is, according to the present investigation, characterized in providing an X-ray powder diffraction pattern (see FIG. 11) exhibiting substantially the following d-values and intensities for the ten most prominent peaks shown in Table M. It will be understood that the relative intensities of peaks may vary according to the orientation of the sample under test and on the type and setting of the instrument used so that the intensities in the X-ray powder diffraction traces included herein are illustrative and not intended to be used for absolute comparison.

TABLE M
Ten most prominent X-Ray Powder Diffraction peaks for magnesium
salt of 1r,4r)-4-(4-(5-(3,4-Difluorophenylamino)-1,3,4-oxadiazole-
2-carboxamido)-3-fluorophenyl)cyclohexanecarboxylic acid.

| d-value | | Relative |
|---|---|---|
| (Angstrom) | Intensity % | Intensity |
| 11.1 | 100 | vs |
| 4.76 | 72.7 | vs |
| 2.36 | 21.7 | s |
| 17.3 | 10.5 | s |
| 3.95 | 8.2 | m |
| 5.2 | 6.1 | m |
| 3.68 | 5.1 | m |
| 5.4 | 5 | m |
| 5.6 | 4.2 | m |
| 3.55 | 3 | m |

vs = very strong;
s = strong;
m = medium

The peaks, identified with d-values calculated from the Bragg formula and intensities, have been extracted from the diffractogram of this salt. The relative intensities have been estimated without any divergence slit conversion and are derived from diffractograms measured with variable slits.

### Example 6

### (1r,4r)-4-(3-Fluoro-4-(5-(2,4,5-trifluorophenylamino)-1,3,4-oxadiazole-2-carboxamido)phenyl)cyclohexanecarboxylic acid

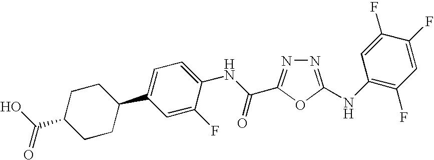

To a suspension of Intermediate 6-1 (0.400 g, 0.79 mmol) in MeOH (10 mL) and THF (5 mL) was added 2N sodium hydroxide (1.975 mL, 3.95 mmol). The resulting solution was stirred at ambient temperature overnight. The reaction mixture was evaporated and the aqueous residue was adjusted to pH with 2M HCl. The precipitate was collected by filtration, washed with water (10 mL) to afford the desired product. The crude product was purified by recrystallised from boiling glacial AcOH (10 mL) to afford the title compound (0.157 g, 41.5%) as a white solid.

¹H NMR (400 MHz, DMSO) δ 1.39-1.53 (4H, m), 1.79-1.88 (2H, m), 1.97-2.02 (2H, m), 2.23-2.31 (1H, m), 2.51-2.57 (1H, m), 7.10 (1H, d), 7.18 (1H, d), 7.44 (1H, t), 7.66-7.73 (1H, m), 8.11-8.20 (1H, m), 10.68 (1H, s), 11.06 (1H, s), 12.01 (1H, s); m/z (M+H)⁺ 479.

### Intermediate 6-1: (1r,4r)-Ethyl 4-(3-fluoro-4-(5-(2,4,5-trifluorophenylamino)-1,3,4-oxadiazole-2-carboxamido)phenyl)cyclohexanecarboxylate

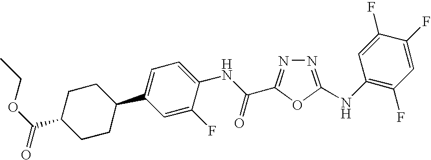

To a solution of Intermediate 1-2 (278 mg, 0.79 mmol) in DMF (10 mL) was added 2,4,5-trifluorophenyl isothiocyanate (150 mg, 0.79 mmol). The resulting mixture was stirred at 45° C. for 45 minutes, 1-(3-Dimethylaminopropyl)-3-ethylcarbodiimide hydrochloride (220 mg, 1.15 mmol) was added and the mixture was stirred at 85° C. for 45 minutes. The reaction mixture was allowed to cool to ambient temperature, water (10 mL) was added and the precipitate was collected by filtration, washed with water (10 mL) and air dried to afford the desired product (401 mg, 100%) as a yellow solid, which was used without further purification.

¹H NMR (400 MHz, DMSO) δ 1.18 (3H, t), 1.44-1.52 (4H, m), 1.82-1.87 (2H, m), 1.96-2.02 (2H, m), 2.31-2.40 (1H, m), 2.54-2.60 (1H, m), 4.06 (2H, q), 7.10 (1H, d), 7.17 (1H, d), 7.43 (1H, t), 7.65-7.73 (1H, m), 8.11-8.19 (1H, m), 10.68 (1H, s), 11.04 (1H, s); m/z (M+H)⁺ 507.

### Example 7

### (1s,4s)-4-(4-(5-(3,4-Difluorophenylamino)-1,3,4-oxadiazole-2-carboxamido)-3-fluorophenyl)cyclohexanecarboxylic acid

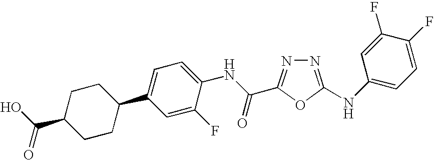

To a suspension of Intermediate 7-1 (0.361 g, 0.74 mmol) in MeOH (10 mL) and THF (5 mL) was added 2N sodium hydroxide (1.850 mL, 3.70 mmol). The resulting solution was stirred at ambient temperature overnight. The reaction mixture was evaporated and the aqueous residue was adjusted to pH 2 with 2M HCl. The suspension was filtered and dried to afford the desired product. This recrystallised from boiling glacial AcOH (10 mL), the solution was allowed to slowly cool and the suspension was filtered and dried to afford the title compound (0.150 g, 44.0%) as a white solid.

¹H NMR (400 MHz, DMSO) δ 1.51-1.64 (4H, m), 1.67-1.73 (2H, m), 2.06-2.12 (2H, m), 2.58-2.64 (2H, m), 7.05 (1H, d), 7.10 (1H, d), 7.31-7.36 (1H, m), 7.43-7.48 (2H, m), 7.65-7.71 (1H, m), 10.66 (1H, s), 11.23 (1H, s), 12.07 (1H, s); m/z (M+H)⁺ 461.

### Intermediate 7-1: (1s,4s)-Ethyl 4-(4-(5-(3,4-difluorophenylamino)-1,3,4-oxadiazole-2-carboxamido)-3-fluorophenyl)cyclohexanecarboxylate

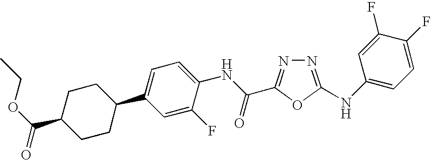

To a solution of Intermediate 7-2 (260 mg, 0.74 mmol) in DMF (10 mL) was added 3,4-difluorophenyl isothiocyanate (152 mg, 0.89 mmol). The resulting mixture was stirred at 45° C. for 45 minutes, 1-(3-dimethylaminopropyl)-3-ethylcarbodiimide hydrochloride (206 mg, 1.07 mmol) was added and the mixture was stirred at 85° C. for 45 minutes. The reaction mixture was allowed to cool to ambient temperature, water (10 mL) was added and the precipitate was collected by filtration, washed with water (10 mL) and air dried to afford the desired product (361 mg, 100%) as a yellow solid, which was used without further purification.

¹H NMR (400 MHz, DMSO) δ 1.21 (3H, t), 1.45-1.75 (6H, m), 2.07-2.12 (2H, m), 2.57-2.64 (1H, m), 2.69-2.72 (1H, m), 4.12 (2H, q), 7.04-7.13 (2H, m), 7.31-7.36 (1H, m), 7.43-7.51 (2H, m), 7.65-7.71 (1H, m), 10.67 (1H, s), 11.22 (1H, s); m/z (M+H)⁺ 489.

### Intermediate 7-2: (1s,4s)-Ethyl 4-(3-fluoro-4-(2-hydrazinyl-2-oxoacetamido)phenyl)-cyclohexanecarboxylate

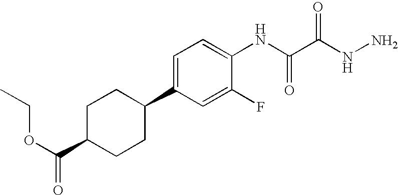

To a solution of Intermediate 7-3 (755 mg, 2.15 mmol) in ethanol (30 mL) was added hydrazine monohydrate (0.115 mL, 2.36 mmol). The resulting suspension was stirred at ambient temperature for 60 minutes. The reaction mixture was evaporated to dryness to afford the title compound (755 mg, 100%) as a yellow solid.

¹H NMR (400 MHz, DMSO) δ 1.20 (3H, t), 1.46-1.72 (6H, m), 2.05-2.11 (2H, m), 2.54-2.62 (1H, m), 2.66-2.73 (1H, m), 4.12 (2H, q), 7.03 (1H, d), 7.09 (1H, d), 7.56 (1H, t); NH protons not seen. m/z (M+H)⁺ 352.

### Intermediate 7-3: (1s,4s)-Ethyl 4-(3-fluoro-4-(2-methoxy-2-oxoacetamido)phenyl)-cyclohexanecarboxylate

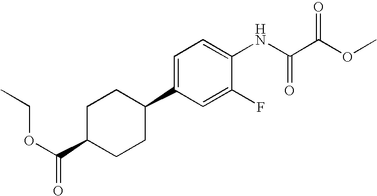

To a solution of Intermediate 7-4 (674 mg, 2.54 mmol) in DCM (20 mL) was added pyridine (0.246 mL, 3.05 mmol) and methyl oxalyl chloride (0.304 mL, 3.30 mmol). The resulting solution was stirred at ambient temperature for 2 hours. The reaction mixture was diluted with DCM (50 mL), and washed with saturated brine (50 mL), the organic layer was dried over MgSO₄, filtered and evaporated to afford the title compound (755 mg, 85%) as a yellow gum, which was used without further purification.

¹H NMR (400 MHz, CDCl₃) δ 1.28 (3H, t), 1.60-1.80 (6H, m), 2.22-2.27 (2H, m), 2.50-2.58 (1H, m), 2.67-2.70 (1H, m), 3.97 (3H, s), 4.19 (2H, q), 6.97-7.02 (2H, m), 8.22 (1H, t), 8.99 (1H, s); m/z (M+Na)⁺ 374.

### Intermediate 7-4: (1s,4s)-Ethyl 4-(4-amino-3-fluorophenybcyclohexane-carboxylate

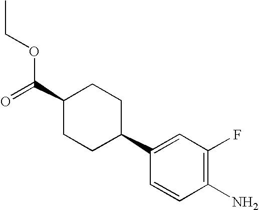

4M Hydrogen chloride in dioxane (15.02 mL, 60.06 mmol) was added to a stirred solution of Intermediate 1-6 (4.39 g, 12.01 mmol) in 1,4-dioxane (5 mL). The resulting mixture was stirred at ambient temperature for 90 minutes. The reaction was incomplete and further 4M hydrogen chloride in dioxane (6 mL, 24 mmol) was added and the solution was stirred at ambient temperature for a further 5 hours. The reaction mixture was evaporated, the mixture adjusted to pH 10 with 2M NaOH and extracted into EtOAc (250 mL). The organic layer was dried over MgSO₄, filtered and evaporated to afford (1s,4s)-ethyl 4-(4-amino-3-fluorophenyl)cyclohexanecarboxylate (3.19 g, 100%) as a brown gum.

¹H NMR (400 MHz, CDCl₃) δ 1.28 (3H, t), 1.52-1.66 (4H, m), 1.69-1.74 (2H, m), 2.17-2.24 (2H, m), 2.41-2.47 (1H, m), 2.64-2.66 (1H, m), 3.57 (2H, s), 4.18 (2H, q), 6.67-6.71 (1H, m), 6.75-6.84 (2H, m); m/z (M+H)⁺ 266.

### Example 8

### (1s,4s)-4-(3-Fluoro-4-(5-(2,4,5-trifluorophenylamino)-1,3,4-oxadiazole-2-carboxamido)phenyl)cyclohexanecarboxylic acid

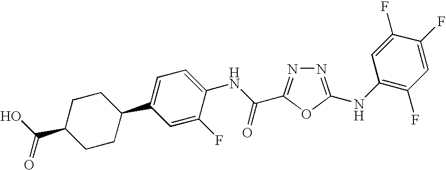

TFA (100 mL) was added to Intermediate 8-1 (7.15 g, 13.38 mmol) and the resulting solution was stirred at 0° C. for 5 minutes and then at ambient temperature for 2 hours. The reaction mixture was evaporated to dryness, the residue was slurried with ether (100 mL) and the mixture was concentrated to afford crude product. The crude solid was suspended in boiling MeCN (90 mL) and the suspension was filtered and dried under vacuum to give 4.83 g of the desired compound as a white solid. The liquors were concentrated and purified by flash silica chromatography, elution gradient 0 to 3% MeOH in DCM. Pure fractions were evaporated to dryness and then suspended in boiling MeCN (˜7 mL), filtered and dried to afford a further 487 mg of desired compound as a white solid. The samples were combined to afford (1s,4s)-4-(3-fluoro-4-(5-(2,4,5-trifluorophenylamino)-1,3,4-oxadiazole-2-carboxamido)phenyl)cyclohexanecarboxylic acid (83%).

¹H NMR (400 MHz, DMSO) δ 1.50-1.64 (4H, m), 1.70-1.76 (2H, m), 2.06-2.10 (2H, m), 2.59-2.65 (2H, m), 7.04-7.12 (2H, m), 7.45 (1H, t), 7.66-7.73 (1H, m), 8.12-8.19 (1H, m), 10.69 (1H, s), 11.05 (1H, s), 12.12 (1H, s); m/z (M+H)⁺ 478.

### Intermediate 8-1: (1s,4s)-tert-Butyl 4-(3-fluoro-4-(5-(2,4,5-trifluorophenyl-amino)-1,3,4-oxadiazole-2-carboxamido)phenyl)cyclohexanecarboxylate

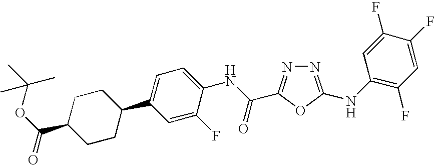

2,4,5-Trifluorophenyl isothiocyanate (2.84 g, 14.99 mmol) was added to a stirred solution of (1s,4s)-tert-butyl 4-(3-fluoro-4-(2-hydrazinyl-2-oxoacetamido)phenyl)cyclohexanecarboxylate (4.74 g, 12.49 mmol) in DMF (60 mL). The resulting solution was stirred at 45° C. for 45 minutes and 1-(3-dimethylaminopropyl)-3-ethylcarbodiimide hydrochloride (3.47 g, 18.11 mmol) was added and the mixture was stirred at 85° C. for 60 minutes. The reaction mixture was allowed to cool to ambient temperature, water (70 mL) was added and the precipitate was collected by filtration, washed with water (50 mL) and dried to afford (1s,4s)-tert-butyl 4-(3-fluoro-4-(5-(2,4,5-trifluorophenylamino)-1,3,4-oxadiazole-2-carboxamido)phenyl)cyclohexanecarboxylate (6.09 g, 91%) as a yellow solid.

¹H NMR (400 MHz, DMSO) δ 1.44 (9H, s), 1.46-1.63 (4H, m), 1.68-1.73 (2H, m), 2.04-2.07 (2H, m), 2.59-2.63 (2H, m), 7.03-7.10 (2H, m), 7.43-7.49 (1H, m), 7.65-7.73 (1H, m), 8.12-8.19 (1H, m), 10.69 (1H, s), 11.03 (1H, s); m/z (M+H)⁺ 535.

### Intermediate 8-2: (1s,4s)-tert-Butyl 4-(3-fluoro-4-(2-hydrazinyl-2-oxoacetamido)-phenyl)cyclohexanecarboxylate

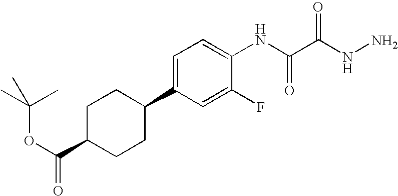

Hydrazine monohydrate (1.044 mL, 13.77 mmol) was added to a stirred solution of Intermediate 8-3 (4.75 g, 12.52 mmol) in ethanol (250 mL). The resulting solution was stirred at ambient temperature for 60 minutes. The reaction mixture filtered and dried under vacuum to afford the desired product (1.98 g) as a white solid, the filtrate was evaporated by two thirds and the suspension was again filtered, combined with the first crop and dried to afford the title compound (4.74 g, 100%) as a white solid.

¹H NMR (400 MHz, DMSO) δ 1.40-1.43 (9H, m), 1.46-1.61 (4H, m), 1.67-1.70 (2H, m), 2.03-2.06 (2H, m), 2.55-2.59 (2H, m), 4.61 (2H, s), 7.00-7.08 (2H, m), 7.57 (1H, t), 10.08 (1H, s), 10.29 (1H, s); m/z (M−H)⁻ 378.

### Intermediate 8-3: (1s,4s)-tert-Butyl 4-(3-fluoro-4-(2-methoxy-2-oxoacetamido)phenyl)-cyclohexanecarboxylate

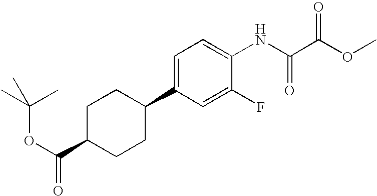

Methyl oxalyl chloride (1.190 mL, 12.94 mmol) was added to a stirred solution of Intermediate 8-4 (2.92 g, 9.95 mmol) and pyridine (0.965 mL, 11.94 mmol) in DCM (100 mL). The resulting solution was stirred at ambient temperature for 1 hour. The reaction mixture was washed sequentially with 1N citric acid (100 mL), saturated brine (100 mL) and the combined aqueous phases were re extracted with DCM (50 mL). The organic extracts were dried over MgSO₄, filtered and evaporated to afford the title compound (3.78 g, 100%) as a pale brown oil which solidified on standing.

¹H NMR (400 MHz, CDCl₃) δ 1.48 (9H, s), 1.58-1.68 (4H, m), 1.70-1.78 (2H, m), 2.18-2.21 (2H, m), 2.50-2.55 (1H, m), 2.60-2.62 (1H, m), 3.98 (3H, s), 6.96-7.02 (2H, m), 8.23 (1H, t), 9.00 (1H, s); m/z (M+Na)⁺ 402.

### Intermediate 8-4: (1s,4s)-tert-Butyl 4-(4-amino-3-fluorophenyl)cyclohexane-carboxylate

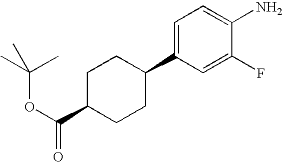

Intermediate 8-5 (4.85 g, 11.34 mmol) and 10% palladium on carbon (400 mg, 3.76 mmol) in EtOAc (100 mL) was evacuated with hydrogen (3 cycles) and then stirred under a balloon of hydrogen at ambient temperature for 90 minutes. The reaction mixture was filtered and evaporated to afford the title compound (2.92 g, 88%) as a colourless oil which solidified on standing.

¹H NMR (400 MHz, CDCl₃) δ 1.48 (9H, s), 1.55-1.67 (4H, m), 1.71-1.73 (2H, m), 2.15-2.19 (2H, m), 2.35-2.45 (1H, m), 2.56-2.58 (1H, m), 6.67-6.71 (1H, m), 6.75-6.78 (1H, m), 6.80-6.84 (1H, m); NH₂ not seen; m/z (M+H)⁺ 294.

### Intermediate 8-5: (1s,4s)-tert-Butyl 4-(4-(benzyloxycarbonylamino)-3-fluorophenyl)cyclohexanecarboxylate

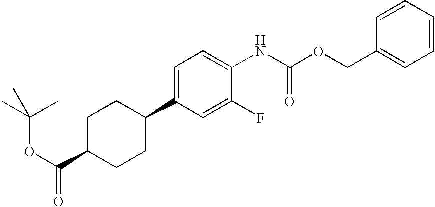

N,N-Dimethylformamide di-tert-butyl acetal (15.58 mL, 65.15 mmol) was added to a stirred solution of Intermediate 8-6 (6.05 g, 16.29 mmol) in toluene (200 mL). The resulting solution was stirred at 85° C. for 3 hours. The reaction was incomplete and further N,N-dimethylformamide di-tert-butyl acetal (6 mL, 32 mmol) was added and the solution was stirred at 85° C. for a further 16 hours (overnight), further N,N-dimethylformamide di-tert-butyl acetal (3 mL) was added of and then allowed to stir at ambient temperature overnight. The reaction mixture was evaporated afford crude product which was purified by flash silica chromatography, elution gradient 0 to 20% EtOAc in isohexane. Pure fractions were evaporated to dryness to afford the title compound (4.15 g, 59.6%) as a pale yellow gum.

¹H NMR (400 MHz, CDCl₃) δ 1.48 (9H, s), 1.50-1.66 (4H, m), 1.68-1.76 (2H, m), 2.17-2.23 (2H, m), 2.46-2.51 (1H, m), 2.58-2.60 (1H, m), 5.21 (2H, s), 6.79 (1H, s), 6.88-6.91 (1H, m), 6.94-6.96 (1H, m), 7.31-7.42 (5H, m), 7.90-7.98 (1H, m); m/z (M+18) 445.

### Intermediate 8-6: (1s,4s)-4-(4-(Benzyloxycarbonylamino)-3-fluorophenyl)-cyclohexanecarboxylic acid

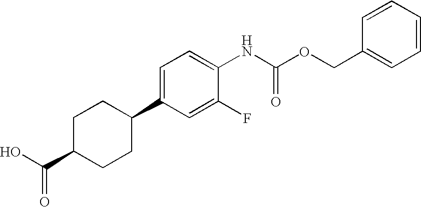

6M hydrochloric acid (20.65 mL, 123.92 mmol) was added to a stirred solution of Intermediate 8-7 (9.90 g, 24.78 mmol) in dioxane (85 mL). The resulting solution was stirred at 80° C. overnight. The reaction mixture was allowed to cool, diluted with EtOAc (300 mL), and washed with saturated brine (200 mL), the aqueous layer was re extracted with EtOAc (200 mL). The organic extracts were combined, dried over MgSO₄, filtered and evaporated to afford crude product. The crude product was purified by flash silica chromatography, elution gradient 10 to 50% EtOAc in isohexane. Pure fractions were evaporated to dryness to afford the title compound (6.22 g, 67.6%) as a white solid which was further dried under vacuum overnight.

¹H NMR (400 MHz, DMSO) δ 1.46-1.61 (4H, m), 1.65-1.70 (2H, m), 2.05-2.08 (2H, m), 2.54-2.61 (2H, m), 5.13 (2H, s), 6.95-7.01 (2H, m), 7.30-7.42 (5H, m), 7.48 (1H, t), 9.28 (1H, s), 12.10 (1H, s); m/z (M−H)⁻ 370.

### Intermediate 8-7: (1s,4s)-Ethyl 4-(4-(benzyloxycarbonylamino)-3-fluorophenyl)-cyclohexanecarboxylate

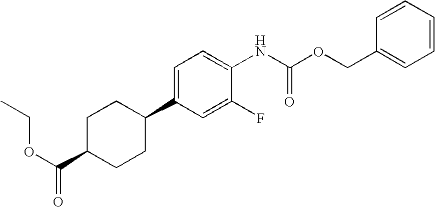

Benzyl chloroformate (4.63 mL, 32.40 mmol) was added to Intermediate 7-4 (8.89 g, 29.46 mmol) and DIPEA (10.26 mL, 58.92 mmol) in DCM (52 mL) at ambient temperature under nitrogen. The resulting solution was stirred at ambient temperature for 1 hour. The reaction mixture was diluted with DCM (50 mL), and washed sequentially with saturated brine (75 mL), 1N citric acid (75 mL), and saturated brine (75 mL). The organic layer was dried over MgSO₄, filtered and evaporated to afford crude product. The crude product was purified by flash silica chromatography, elution gradient 0 to 20% EtOAc in isohexane. Pure fractions were evaporated to dryness to afford the title compound (9.90 g, 84%) as a pale yellow oil which solidified on standing.

¹H NMR (400 MHz, CDCl₃) δ 1.28 (3H, t), 1.57-1.67 (4H, m), 1.71-1.77 (2H, m), 2.19-2.25 (2H, m), 2.47-2.53 (1H, m), 2.66-2.67 (1H, m), 4.18 (2H, q), 5.21 (2H, s), 6.80 (1H, s), 6.88-6.92 (1H, m), 6.95 (1H, s), 7.31-7.42 (5H, m), 7.95 (1H, t); m/z (M+Na)⁺ 422.

1. A compound of formula (I), or a pharmaceutically-acceptable salt thereof, 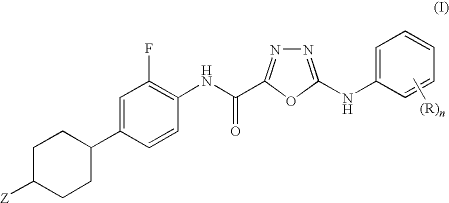
wherein:
n is 0, 1, 2 or 3;
R is independently selected from fluoro, chloro, bromo, trifluoromethyl, methoxy, difluoromethoxy and trifluoromethoxy;
Z is carboxy, hydroxyl, hydroxymethyl, —CONRbRc, —SO₃H, —S(O)₂NHR¹³, S(O)₂NHC(O)R¹³, —C₂S(O)₂R¹³, —C(O)NHS(O)₂R¹³, —C(O)NOH, —C(O)NHCN, —CH(CF₃)OH, C(CF₃)₂OH, —P(O)(OH)₂ or a 5-membered heterocyclic ring selected from the group consisting of 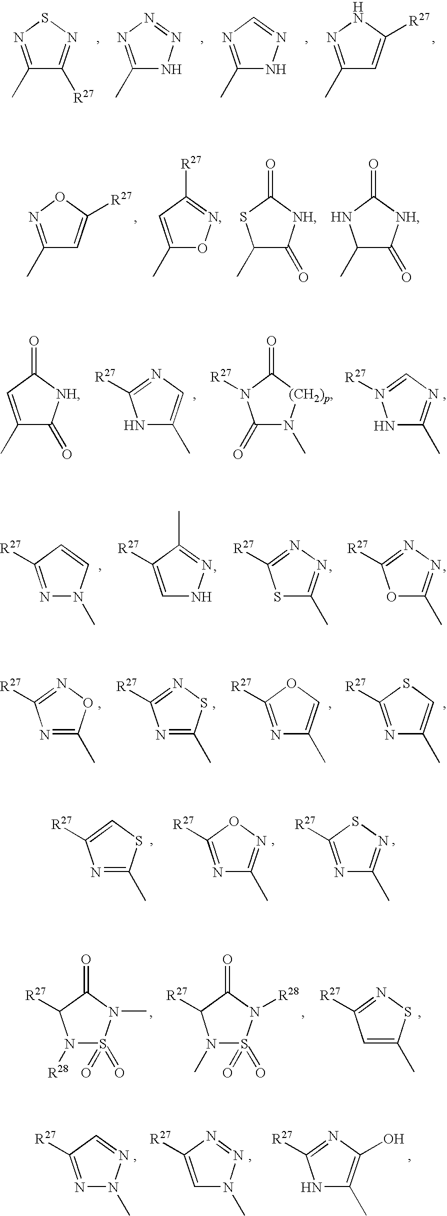 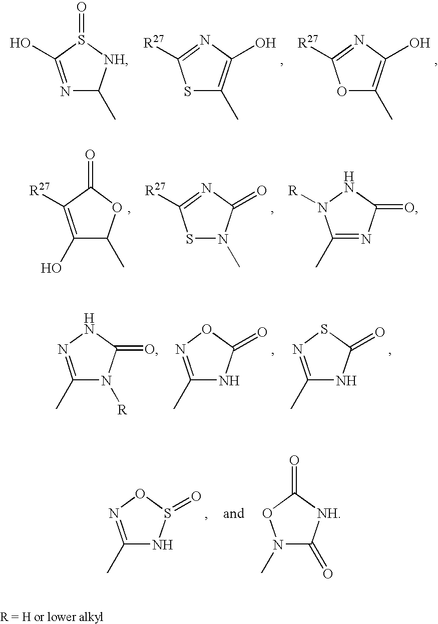
p is 1 or 2:
R²⁷ and R²⁸ are independently selected from hydrogen, hydroxy, (1-6C)alkoxy, thiol, (1-6C)alkylthio, —C(O)R²⁹, —S(O)R³⁰, —SO₂R³¹, —NR³²R³³, —NHCN, halogen and trihalomethyl;
R²⁹, R³⁰ and R³¹ are —OR³⁴, (1-6C)alkyl, —NR³²R³³ or trihalomethyl;
R³² and R³³ are independently selected from hydrogen, (1-6C)alkyl, —SO₂R³⁴ and —COR³⁵;
R³⁵ is (1-6C)alkyl or trihalomethyl;
R³⁴ is hydrogen, (1-6C)alkyl or trihalomethyl; and
R¹³ is selected from hydrogen, (1-6C)alkyl, hydroxy, halo, amino, cyano, ((1-3C)alkyl)CONH—, carboxy, (1-6C)alkoxy, (1-6C)alkoxycarbonyl, carbamoyl, N-((1-6C)alkyl)carbamoyl, halo((1-6C)alkyl) (such as trifluoromethyl), (1-6C)alkylsulphonyl or (1-6C)alkylsulphinyl;
Rb and Rc are independently selected from hydrogen and (1-4C)alkyl, which (1-4C)alkyl group may be optionally substituted by carboxy, —SO₃H, —S(O)₂NHR¹³, S(O)₃NHC(O)R¹³, —CH₂S(O)₂R¹³, —C(O)NHS(O)₂R¹³, —C(O)NHOH, —C(O)NHCN, —CH(CF₃)OH, C(CF₃)₂OH, —P(O)(OH)₂ or a 5-membered heterocyclic ring as defined above.

2. The compound of formula (IA) as claimed in claim 1, or a pharmaceutically-acceptable salt thereof, 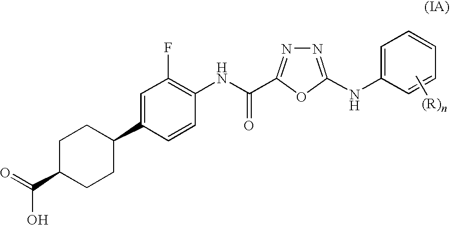
wherein n and R are as defined in claim 1.

3. The compound of formula (IB) as claimed in claim 1, or a pharmaceutically-acceptable salt thereof, 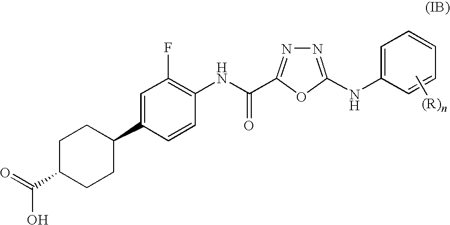
wherein n and R are as defined in claim 1.

4. The compound as claimed in claim 1, or a pharmaceutically-acceptable salt thereof, wherein n is 2 or 3 and R is fluoro.

5. The compound as claimed in claim 1 selected from
(1r,4r)-4-(3-Fluoro-4-(5-(4-(trifluoromethyl)phenylamino)-1,3,4-oxadiazole-2-carboxamido)phenyl)cyclohexanecarboxylic acid;
(1r,4r)-4-(4-(5-(4-(Difluoromethoxy)phenylamino)-1,3,4-oxadiazole-2-carboxamido)-3-fluorophenyl)cyclohexanecarboxylic acid;
(1r,4r)-4-(3-Fluoro-4-(5-(4-(trifluoromethoxy)phenylamino)-1,3,4-oxadiazole-2-carboxamido)phenyl)cyclohexanecarboxylic acid;
(1r,4r)-4-(4-(5-(3-Chlorophenylamino)-1,3,4-oxadiazole-2-carboxamido)-3-fluorophenyl)cyclohexanecarboxylic acid;
(1r,4r)-4-(4-(5-(3,4-Difluorophenylamino)-1,3,4-oxadiazole-2-carboxamido)-3-fluorophenyl)cyclohexanecarboxylic acid;
(1r,4r)-4-(3-Fluoro-4-(5-(2,4,5-trifluorophenylamino)-1,3,4-oxadiazole-2-carboxamido)phenyl)cyclohexanecarboxylic acid;
(1s,4s)-4-(4-(5-(3,4-Difluorophenylamino)-1,3,4-oxadiazole-2-carboxamido)-3-fluorophenyl)cyclohexanecarboxylic acid and
(1s,4s)-4-(3-Fluoro-4-(5-(2,4,5-trifluorophenylamino)-1,3,4-oxadiazole-2-carboxamido)phenyl)cyclohexanecarboxylic acid;
or a pharmaceutically-acceptable salt of any of these.

6. The compound (1r,4r)-4-(4-(5-(3,4-Difluorophenylamino)-1,3,4-oxadiazole-2-carboxamido)-3-fluorophenyl)cyclohexanecarboxylic acid, as claimed in claim 1, of formula (ID) 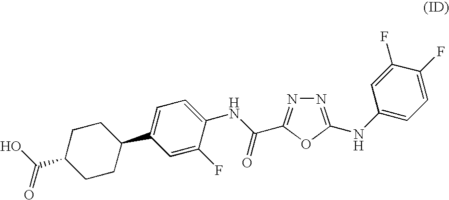
or a pharmaceutically-acceptable salt thereof.

7. The compound (1r,4r)-4-(4-(5-(3,4-Difluorophenylamino)-1,3,4-oxadiazole-2-carboxamido)-3-fluorophenyl)cyclohexanecarboxylic acid, as claimed in claim 1, of formula (ID)

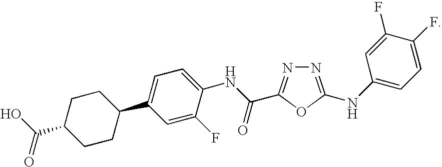

8. The compound (1r,4r)-4-(4-(5-(3,4-Difluorophenylamino)-1,3,4-oxadiazole-2-carboxamido)-3-fluorophenyl)cyclohexanecarboxylic acid, as claimed in claim 1, characterized by an X-ray powder diffraction pattern with specific peaks at about 2-theta equal to 16.2° and 27.6°.

9. The compound (1r,4r)-4-(4-(5-(3,4-Difluorophenylamino)-1,3,4-oxadiazole-2-carboxamido)-3-fluorophenyl)cyclohexanecarboxylic acid, as claimed in claim 1, in a form selected from a form characterized by an X-ray powder diffraction pattern with specific peaks at the following d-values
(a) 17.3, 5.2 and 3.66 Angstroms;
(b) 5.5, 13.3, 3.50, 3.33 and 6.6 Angstroms;
(c) 4.72, 5.1, 3.45, 3.43 and 15.6 Angstroms;
(d) 4.78, 3.38, 4.12, 5.29, 6.2, 3.7 and 21.6 Angstroms;
(e) 3.75, 4.43 and 14.2 Angstroms or
(f) 13.8, 5.5, 3.48 and 3.22 Angstroms.

10-13. (canceled)

14. A method of treating diabetes mellitus and/or obesity in a warm-blooded animal, in need of such treatment comprising administering to said animal an effective amount of a compound according to claim 1, or a pharmaceutically-acceptable salt thereof.

15. A pharmaceutical composition comprising a compound of formula (I) as claimed in claim 1 or a pharmaceutically-acceptable salt thereof, in association with a pharmaceutically-acceptable excipient or carrier.

16. A method for inhibiting DGAT1 activity in a warm-blooded animal in need of such treatment comprising administering to said animal an effective amount of a compound of formula (I) as claimed in claim 1 or a pharmaceutically-acceptable salt thereof.

17. The method as claimed in claim 14 or claim 16, wherein the warm-blooded animal is a human being.
